(19) United States
(12) Patent Application Publication
(10) Pub. No.: US 2004/0009178 A1
(43) Pub. Date: Jan. 15, 2004

(54) Immunotherapeutics for biodefense

(76) Inventors: Katherine S. Bowdish, Del Mar, CA (US); Shana Frederickson, Solana Beach, CA (US); Martha A. Wild, San Diego, CA (US); Toshiaki Maruyama, La Jolla, CA (US); Mary Jean Nolan, San Diego, CA (US)

Correspondence Address: Mark Farber, Esq., Alexion Pharmaceuticals, Inc., 352 Knotter Drive, Cheshire, CT, 06410, US

(21) Appl. No.: 10/364,743

(22) Filed: Feb. 11, 2003

Related U.S. Application Data
(60) Provisional application No. 60/356,086, filed on Feb. 11, 2002.
(60) Provisional application No. 60/376,408, filed on Apr. 29, 2002.
(60) Provisional application No. 60/414,053, filed on Sep. 27, 2002.
(60) Provisional application No. 60/428,807, filed on Nov. 25, 2002.

(51) Int. Cl. A61K 39/40; C07K 16/12

(52) U.S. Cl. 424/164.1; 530/388.15; 530/388.4

(57) ABSTRACT

Human neutralizing antibodies (full-length or functional fragments) are useful as anti-toxins or anti-infectives with respect to infective agents such as, for example, anthrax, botulinum, smallpox, Venezuelan equine encephalomyelitis virus (VEEV), West Nile virus (WNV) and the like.

### RELATED APPLICATIONS

This applications claims priority to U.S. Provisional Application Nos. 60/356,086, 60/376,408 and 60/428,807 filed on Feb. 11, 2002, Apr. 29, 2002 and Nov. 25, 2002, respectively.

### BACKGROUND

1. Technical Field

This disclosure relates to human neutralizing antibodies (full-length or functional fragments) useful as anti-toxins or anti-infectives with respect to infective agents such as, for example, anthrax, botulinum, smallpox, Venezuelan equine encephalomyelitis virus (VEEV), West Nile virus (WNV) and the like.

2. Background of Related Art

Concerns over our capacity to prevent and treat anthrax infection have been high following the recent acts of terrorism in the US. Though a vaccine for anthrax exists, it consists of six spaced inoculations, requires a yearly booster, and produces unpleasant side effects in most vaccinees. This has kept it from widespread use, and is still a major drawback to inoculating the general public. The anthrax vaccine presently in use is made by Bioport (Lansing, Mich.) through a process that involves purifying the protective antigen out of a lysate of Bacillus anthracis. It appears that this is still the Sterne live vaccine strain, though there are other strains lacking the LF and EF that might be used to generate this vaccine, as well as high yielding recombinant Bacillus subtilis strains that could be used. Presumably the difficulties of testing and comparing such additional vaccines, and the small market, have hindered actual testing, licensing and production. The side effects of the present vaccine, its connection in the minds of many with Gulf War Syndrome, and the possibility that large scale vaccination may now be desirable in the face of terrorist threats suggests that improvements in the present vaccine will now be pursued. As long as there is no reasonable vaccine, treatment for exposure remains the primary response to acts of terrorism.

The recent anthrax exposures in the United States from contaminated letters have all involved strains of anthrax susceptible to antibiotics, but tragically, a number of people died due to delayed diagnosis. In light of this, it would be useful to have a treatment for anthrax exposure that could prevent illness and death and allow antibiotic therapy and/or adaptive immunity additional time to be effective.

The primary cause of death from anthrax is the reaction of the body to two related toxins produced by the bacteria. These both contain a processed protein called PA63 that binds as PA83 to cellular receptors, whereupon it is processed to PA63. The receptor on human cells for anthrax toxin, the ATR (anthrax toxin receptor) was recently identified. See Bradley et al., Nature, Vol 414, Nov. 8, 2001. PA63 then forms a heptamer that is capable of binding with either the EF protein (edema factor) or the LF protein (lethal factor). Endosomal internalization of heptamerized PA63 and bound EF and/or LF facilitates the introduction of the EF and LF toxins into the cell. Acidification of the endosomal vesicle causes the PA heptamer to form a pore through which the EF and/or LF can enter the cytosol, where they exert their toxic effects. None of the three components, EF, LF, or PA, can cause illness by itself.

Several lines of evidence indicate that it is possible to prevent receptor binding of PA or to obstruct the interaction of EF and LF with PA. The vaccine itself uses the purified PA moiety alone to create antibodies that are protective. Little et al. (Infect. Immun. 65:5171-5175 (1997)) passively administered PA antibodies to guinea pigs that then showed protection against subsequent anthrax infection—70% protection for polyclonal antibodies, and a two day delay of death for one PA63 monoclonal. Single-chain antibody fragments (scFvs) have also been used to inhibit receptor binding by PA. Cirino et al. (Infect. Immun. 67:2957-2963 (1999)) identified a number of scFvs from a naïve human library that bound PA83. They then used these in a cell-based assay in which PA32, a truncated version of PA63, was fused with EGFP, and was taken up by cells in a similar manner to PA63. The fluorescence of EGFP could then be used to monitor the effect of these scFvs against PA32-EGFP in cellular uptake. One scFv was identified which could prevent the uptake of the PA32-EGFP by the cell. Mourez et al. (Nature Biotech. 19:958-961 (2001)) created a polyvalent peptide inhibitor against the anthrax toxin that binds to the PA63 heptamer at or near the EF/LF site. They used cell-based assays to demonstrate that this inhibitor can protect cells against PA/LF toxicity. They also showed that rats could be challenged with 10 times the minimum lethal dose of PA and LF and still be protected when the inhibitor was introduced 3-4 minutes after challenge.

These data all suggest that it should be possible to develop a therapeutic human combinatorial antibody for combination therapy with antibiotics in patients with late diagnoses of anthrax infection.

Venezuelan equine encephalomyelitis virus (VEEV) is a mosquito-borne alphavirus which can be transmitted to both equine and human hosts. Whereas infection of horses and donkey populations can result in large mortalities, natural human infection usually consists of fever, chills, malaise, and severe headache with only 1-4% of people progressing to severe encephalitis. However, VEEV has been classified as a “Category B” critical biological agent by the CDC due to its low human infective dose, easy production, and ability to be aerosolized. Potentially, aerosolized VEEV could be used as an effective bioweapon using forms of VEEV that are known to be highly infectious and that can easily gain direct access to the central nervous system via the olfactory tract. Once replication of the virus occurs in the CNS, encephalitis is a serious risk. Unfortunately, treatment of VEEV infection is limited to supportive care.

There are investigational vaccines available against VEEV, although their use is limited to laboratory workers at risk and military troops. A live attenuated vaccine, TC-83 (FDA Investigational New Drug #142) (Pittman et al., Vaccine 14, pp337-343 (1996)), has been used in both these settings. This vaccine was established by serial passage of the virulent Trinidad donkey virus in tissue culture. TC-83 virus elicits VEEV-specific neutralizing antibodies in most humans and equines. (Kinney et al., Virology 170, 19-30 (1989)). In laboratory animals, the vaccine was able to produce immunity to subcutaneous or airborne challenge with virulent VEEV strains (Phillpotts, Vaccine 17, pp2429-2435(1999)). However, up to 18% of human vaccinees fail to develop protection from the initial vaccinations. In addition, the vaccine has a relatively high rate of reactogenicity (25%). One recent report states that TC-83 is no longer available for human use (Phillpotts et al., Vaccine 20, p1497-1504 (2002)). Concerns over TC-83 prompted the development of an inactivated vaccine, C-84. However, C-84 did not produce protection against aerosol challenge with virulent strains of the virus in animal models (Pittman et al., Vaccine, 14, pp337-343 (1996).). As a result, C-84 is not used as a primary immunogen for laboratory workers, rather its usefulness is as a follow up vaccine for non-responders to TC-83 or as a booster where it serves as a recall antigen. There is, therefore, an urgent need for anti-VEEV therapies, such as potent neutralizing anti-VEEV antibodies.

VEEV is an enveloped virus, where the envelope and capsid structures are separated by a lipid bilayer and are thought to interact through the membrane-spanning tail of the E2 glycoprotein. Similar to Sindbis virus, VEEV has virion protein spikes organized as trimers of E1/E2 heterodimers (Paredes, et al., J. Virology, 75, ppp9532-9537 (2001); Phinney, et al. J Virology, 74, pp5667-5678 (2000)). The epitopes present on E1 (gp50) and E2 (gp56) that may be involved in the critical neutralization sites have been studied using monoclonal Abs (Mathews and Roehrig, J. Immunology, pp2763-2767 (1982)). Site E2^{c} is present at the tip of the E2 spike and believed to be the neutralizing (N) epitope. Additional epitopes have also demonstrated neutralizing activity and may have a close structural relationship with the E2^{c} site.

The mouse model of VEEV infection is believed to follow a pathogenesis of disease that is similar to that in humans. Passive transfer of neutralizing Ab prior to viral challenge has effectively prevented death in these normal mice (see for example, Roehrig and Mathews, Virology (1985) 142, pp 347-356; Phillpotts, et al., Vaccine (2002) 20, 1497-1504). Non-neutralizing Abs have also shown protection in i.p. or i.v. viral challenge of mice (Hunt and Roehrig, Vaccine (1995) 13, pp281-288; and Hunt et al., Virology (1991) 185, 281-290). Although the mechanism of non-neutralizing Ab protection from viral challenge is not known, it is surmised that they may act by delaying viral replication and in doing so allow the host immune system time to respond to and control the viral infection (Hunt et al., Virology (1991) 185, 281-290). An effective therapy for humans against airborne exposure to VEEV may require a faster mode of action, such as direct neutralization of the virus at, or close to, the time of exposure. Of particular concern is the ability of the neutralizing Ab to prevent the spread of VEEV to the brain. In this regard, it is significant that administration of a neutralizing Ab to mice up to 24 hours after airborne viral challenge showed protective effects (Phillpotts, et al., Vaccine, 20, 1497-1504 (2002)).

The murine antibodies described in these and similar studies might be of use in the prevention and treatment of VEEV infection in humans. However, rodent antibodies are highly immunogenic in humans and therefore limited in their clinical applications, especially when repeated administration is required for therapy. A process termed antibody humanization can be used to decrease the immunogenicity of a rodent antibody by replacing most of the rodent antibody with human antibody regions while striving to maintain the original antigenic specificity. However, this undertaking is usually time-consuming and costly and does not rule out the possibility of an immunogenic response to the humanized Ab. Antibodies that are fully human and target neutralizing epitopes on VEEV are the most desirable therapeutic candidates, as they pose the best chance of an effective block of viral infection and present the least risk of being immunogenic.

Botulinum neurotoxin is one of the most potent bacterial toxins known, with an LD50 for humans of 1 ng/kg. The toxin is produced by the bacteria Clostridium botulinum, as well as by several other Clostridium species, and seven serotypes of toxin (A through G) have been recognized. On a molecular level, the toxin is produced as a 150 kDa protein that is cleaved by exposure to proteases to generate two chains that remain associated: a light chain, of about 50 kDa, and a heavy chain, of 100 kDa. The heavy chain contains the domain responsible for binding to neuronal cells, while the light chain contains a zinc-dependent endoprotease domain that enters the neuronal cytosol. Once inside, this endoprotease exerts its toxic effect by proteolytic cleavage of synaptic proteins, including synaptobrevins, syntaxin and SNAP-25. Destruction of these proteins inhibits neurotransmission and results in a progressive paralysis and death.

Antibodies against botulinum neurotoxin have been shown to be protective in passive and active immunization models. The PBT vaccine, consisting of serotypes A through E, is currently made available by the Department of Defense and the Centers for Disease Control to people at risk for exposure to botulinum neurotoxin. Serotypes D and G are rarely encountered in natural human infections, though serotype F is common, and is lacking in the PBT vaccine. The possibility of serotypes D and G being utilized in a bioterrorist attack should not, however, be overlooked. For natural infections, polyclonal antibody preparations have been successfully used as immunotherapeutics, but they must be given early in infection to minimize the entry of toxin into the neuronal cells. Several polyclonal immunoglobulin preparations are available as immunotherapeutics: an equine trivalent (A, B, and E) preparation, an equine heptavalent preparation with the Fc portion of the immunoglobulins removed by proteinase cleavage, and a human immunoglobulin preparation (hBIG) obtained from donors vaccinated with the PBT vaccine. Both equine preparations have had difficulties with hypersensitivity reactions in treated individuals. The human preparation is well-tolerated and effective, but it is in short supply and only useful against five of the seven serotypes. Even for natural infections, it would be useful to have a ready supply of fully human neutralizing antibodies to all the serotypes of botulinum neurotoxin. The heightened awareness of our vulnerability to biological terrorism following the intentional anthrax release of 2001 makes it even more critical to develop such immunotherapeutics.

Since the declaration of smallpox eradication in May 1980 (Fenner et al., 1998) and the cessation of vaccination programs, immunity has waned among those vaccinated, and those born since 1980 are unvaccinated. The world-wide lack of immunity dramatically increased the threat of a deliberate release of variola virus, the causative agent of smallpox, as a bioweapon. The variola virus has characteristics that make it particularly suitable for biological warfare. The virus can spread from person to person by the respiratory route or by direct contact. An aerosol release of the virus can disseminate widely, because of its considerable stability in aerosol form and because the infectious dose can be very small (Wherle et al., 1970). There is no specific treatment for the disease. There is also a threat that a large quantity of infectious virus may be missing. Alibek (Alibek, 1999), a former deputy director of the Soviet Union's civilian bioweapons program, reported that beginning in 1980, the Soviet government initiated a bioweapons program and developed a method to produce many tons of variola virus annually for transport in bombs and ballistic missiles. With the decline of financial support for and the discontinuation of the Soviet civilian biowarfare program in 1992, experienced scientists, equipment, and materials may have been transferred into other countries. The reported epidemics in Asia had a mortality-rate of 30% or more (Fenner et al., 1998). Today, with a more susceptible and highly mobile population, the virus can spread very rapidly and widely throughout the country and the world.

Variola virus is a DNA virus, a member of the family Poxyiridae and the genus orthopoxvirus (Fenner et al., 1988) that includes vaccinia, monkeypox virus, and several other animal poxviruses that cross-react serologically. Only variola virus can readily transmit from person to person (reviewed in Breman and Henderson, 2002). DNA sequence analysis revealed that variola and vaccinia viruses are closely related (Massung et al., 1994). The infectious dose of variola virus is believed to be very low, only a few virions (Wherle et al., 1970). It transcribes and replicates its genome and assembles progeny virions entirely within the cytoplasm of infected cells (reviewed in Moss, 1996). Four types of infectious forms are produced: intracellular mature virus (IMV), the intracellular enveloped virus (IEV), the cell-associated enveloped virus (CEV), and the extracellular enveloped virus (EEV) (reviewed in Moss 1996). IMV is the major form that remains in the cytoplasm. EEV represents a minor fraction of infectious particles but is the biologically relevant form in terms of long-range dissemination and spread of the virus in vitro and in vivo (Payne, 1980; Smith and Vanderplasschen, 1998; Law and Smith, 2001). It has been shown that an immune response against EEV but not IMV is necessary for protection against orthopoxvirus infection (Appleyard et al., 1971; Boulter, 1969; Boulter and Appleyard, 1973; Boulter et al., 1971; Ichihashi et al., 1971; Morgan, 1976; Payne, 1980; Payne and Kristensson, 1985; Turner and Squires, 1971). Six genes are reported to encode ten proteins for EEV outer envelope (Payne, 1978; Payne, 1979). They are A33R (gp22-28) (Roper et al., 1996), A34R (gp22-24) (Duncan and Smith, 1992), A36R (p45-50) (Parkinson and Smith, 1994), A56R (gp86, a heavily glycosylated hemagglutinin) (Payne and Norrby, 1976; Shida, 1986), B5R (gp42) (Isaacs et al., 1992; Englestad et al., 1992), and F12L or F13L (p37) (Hirt et al., 1986; Blasco and Moss, 1991). Recently A36R protein was found to be absent in the CEV and EEV particles (van Eijl et al., 2000). Envelope proteins of IMV are A27L (p14) (Rodriguez and Esteban, 1985), D8L (p32) (Maa et al., 1990; Niles and Seto, 1988), A17L (p21) (Rodriguez et al., 1995), and L1R (M25, a myristylated virion protein) (Franke et al., 1990). A27L, A17L and L1R are implicated in the fusion and penetration of IMV (Ichihashi and Oie, 1996).

The smallpox vaccine, manufactured from the vaccinia virus, was the first vaccine ever produced. The current stockpile consists of a live vaccinia virus that was grown on the skin of calves. In the United States, the reserve supply is limited; there is just enough to vaccinate 6 to 7 million people. None of the other countries have enough doses to cover their population if an outbreak occurs. Smallpox vaccination is also associated with more severe adverse effects than any other type of vaccination, which was one of the reasons for ending vaccination after eradication (Ober et al., 2002). Presently, it is recommended for use only in suspected cases and not for mass vaccination by World Health Organization and United States, Centers for Disease Control and Prevention (Smallwood et al., 2002). Vaccination with vaccinia virus is effective in preventing smallpox for at least five years and may prevent or modify infection for a much longer period, but this varies greatly from person to person.

There is general agreement that neutralizing antibodies play an important role in immunity against orthopox viruses, particularly in the prevention of reinfection and dissemination of infection. The benefit of vaccinia immune globulin (VIG) in preventing infection or controlling adverse effects from vaccinia immunization have been clearly documented (Kempe, 1960, Kempe et al., 1961, Hobday, 1962). Polyclonal antiserum against the recombinant B5R protein inhibited EEV infection (Galmiche et al., 1999). Mice vaccinated with B5R protein were protected against a lethal challenge with vaccinia virus that is likely to be mediated by neutralizing antibodies. Protein A33R but not A34R and A36R was also protective in active and passive immunization but protection did not correlate with antibody titers and anti-A33R antibodies did not neutralize EEV in vitro. The authors stated the protection probably involves a mechanism different from simple antibody binding (Galmiche at al., 1999, Schmaljohn et al., 1999). Prophylactic as well as therapeutic administration of mouse neutralizing antibody against the trimeric 14 kDa protein (A27L, p14) of vaccinia virus localized in the membrane of the IMV effectively controlled the replication of the virus in mice (Ramirez et al., 2002). DNA vaccination with L1R and A33R genes protected mice against a lethal virus challenge with neutralizing antibodies to L1R and A33R (Hooper et al., 2000).

As described in the recent issue of Emerging Infectious Diseases (Casadeval, 2002), the only available countermeasure that can provide immediate immunity against a biological agent is passive immunization with antibodies. Vaccine takes time to induce protective immunity and depends on the host's ability to mount an immune response, whereas passive immunization can theoretically confer protection regardless of the immune status of the host. Low cytotoxicity and highly specific activity are among the advantages of passive immunization over other measures of postexposure treatment.

Identification of immune donors with good serum neutralizing activity and the construction of combinatorial antibody libraries from the bone marrow of such donors is a logical approach for the isolation of a large panel of highly specific neutralizing antibodies to viral infection (Burton et al.,1991; Barbas et al., 1992; Williamson et al., 1993; Burioni et al., 1994; Maruyama et al., 1999; Maruyama et al., 2002). The selection of libraries on recombinant envelope proteins containing neutralizing epitopes is straightforward. Unlike mouse antibodies, human antibodies are non-immunogenic and once their efficacy is fully characterized in susceptible animals, they can be safely administered to patients.

It would be desirable to identify antibodies that neutralize infective agents of the types that may be employed in bio-warfare. It would also be advantageous if these bio-defense antibodies could be derived from a single antibody library.

### SUMMARY

Using phage display technologies and messenger RNA derived from lymphoid cells of vaccinated or convalescent humans, it is possible in accordance with the methods described herein to rapidly identify panels of antibody fragments (Fabs) that bind to antigens from infective agents. The strength of the interaction of these Fabs with antigen can be determined by studying their binding kinetics using surface plasmon resonance. These human Fabs can be readily converted to full-length IgG by subcloning into appropriate mammalian expression vectors containing the remaining constant region domains. Testing of Fabs or antibodies from these panels in viral or toxin inhibition studies in vitro and in vivo in small animal models can then identify a subset of neutralizing antibodies that will be suitable for continuation to pre-clinical and clinical testing. These antibodies may then be used as immunotherapeutics in the treatment of individuals infected with or exposed to any of the above agents, or may be used prophylactically in individuals expected to be at risk for exposure.

Thus, in one aspect, an antibody library is described from which antibodies or functional fragments thereof can be identified, isolated and produced in large quantities to neutralize or prevent infection by an infective agent.

In another aspect, heterodimeric antibodies are described which are effective in treating anthrax infection. The heterodimeric antibodies are selected from an antibody library. The library is preferably generated from an immunized human source. The heterodimeric antibodies bind to and disable the activity of a molecule involved in anthrax infection, such as, for example, the anthrax protective antigen or the EF or LF proteins and thereby inhibit toxin activity by interfering with the processes involved in toxin introduction to the cell. These processes include but are not limited to the following: PA83 binding to receptor, PA83 processing to PA63, PA63 interaction to form a prepore complex, EF or LF binding to the prepore, prepore conformational changes permitting membrane translocation of EF or LF, or EF or LF translocation through the pore. This interference is such that the toxic effects associated with uptake of these proteins by cells in the body are slowed or eliminated. In particularly useful embodiments, the heterodimeric antibodies have an affinity of at least 1×10⁻⁸ M for a molecule involved in anthrax infection. In another embodiment, these antibodies can be used as diagnostic reagents.

In another aspect, antibodies or functional fragments of antibodies that neutralize Botulinum are described.

In another aspect, antibodies or functional fragments of antibodies that neutralize Variola virus (Small Pox)/Vaccinia virus are described.

In another aspect, antibodies or functional fragments of antibodies that neutralize Venezuelan Equine Encephalomyelitis Virus (VEEV) are described.

In another aspect, antibodies or functional fragments of antibodies that neutralize West Nile virus (WNV) are described.

In another aspect, antibodies or functional fragments of antibodies that neutralize Dengue are described.

In another aspect, methods of prophylactically administering antibodies or functional fragments of antibodies are described to prevent infection by an infective agent.

In another aspect, methods of administering antibodies or functional fragments of antibodies are described to treat infection by an infective agent.

### BRIEF DESCRIPTION OF THE DRAWINGS

FIG. 1 is a table summarizing the exposure history of individuals suitable as a source of tissue for library generation in accordance with preferred embodiments of the present disclosure.

FIG. 2 shows titers of bone marrow and blood donors to PA83 antigen of Anthrax.

FIG. 3 shows the sequence analysis of the VH positive reactivity to PA63 and PA83.

FIG. 4 shows the sequence analysis of the VK positive reactivity to PA63 and PA83.

FIG. 5 shows the sequence analysis of the VL positive reactivity to PA63 and PA83.

FIG. 6 shows sequences of variant human kappa light chains of antibodies to the anthrax proteins PA83 and PA63.

FIG. 7 shows sequences of variant human lambda light chains of antibodies to the anthrax proteins PA83 and PA63.

FIGS. 8A-8C show sequences of variant human kappa heavy chains of antibodies to the anthrax proteins PA83 and PA63.

FIG. 9 shows neutralization of Anthrax toxin activity by purified Fabs.

FIG. 10 shows the percent protection (compared to toxin alone) for seven serially diluted Fabs.

FIG. 11 shows Western blots demonstrating the ability of Fabs produced in accordance with the methods described herein to react with linear epitopes on PA63 and/or PA83. All of the five anti-PA83 Fabs tested appear to bind to linear epitopes on PA83 while the anti-PA63 antibody, in contrast does not bind to denatured PA63, and shows what appears to be faint, presumably non-specific binding to PA83.

FIG. 12 shows an ELISA titration of selected Fabs on PA83 and PA63. Cleavage to PA63 dramatically alters the binding of FML8E and F9L6R2, but FMK7C binds equally well to both forms. F951 L631 D binds only to PA63. Maximum binding seen is ¼ that of FMK7C, suggesting that only a portion of the PA63 material is in a form with which F951L631D can interact.

FIG. 13 shows the result of testing wherein a his tagged version of Fab FML8E was used in competition with other untagged Fabs to assess epitope specificity.

FIG. 14 shows serum reactivity on immobilized TC-83 antigen of VEEV.

FIGS. 15A through 15D show the results of screening of Fab clones from four libraries (951 K. 951 L, 1037K and 1037L) for binding to immobilized TC-83 of VEEV.

FIG. 16 shows direct titration of purified human Fabs on immobilized TC-83 antigen of VEEV.

FIG. 17 shows competition of the human Fabs against the murine Fab mHy4 (3B4C-4) for binding to immobilized TC-83 antigen of VEEV or BSA.

FIGS. 18A and 18B show the sequences for fully-human Fabs produced in accordance with this disclosure that neutralize VEEV.

### DETAILED DESCRIPTION OF PREFERRED EMBODIMENTS

The human antibodies in accordance with this disclosure can be whole antibodies or antibody fragments. The antibodies can be heterodimeric or single chain antibodies. The term “heterodimeric” means that the light and heavy chains of the antibody or antibody fragment are bound to each other via disulfide bonds as in naturally occurring antibodies. Single chain antibodies have the light and heavy chain variable regions of the antibody connected through a linker sequence.

The present human antibodies are identified by screening an antibody library. Techniques for producing and screening an antibody library are within the purview of one skilled in the art. See, Rader and Barbas, Phage Display, A Laboratory Manual, Cold Spring Harbor Laboratory Press, Cold Spring Harbor, N.Y. (2000), U.S. Pat. No. 6,291,161 to Lerner et al. and copending U.S. Provisional Application Nos. 60/323,455 and 60/323,400, the disclosures of which are incorporated herein in its entirety by this reference.

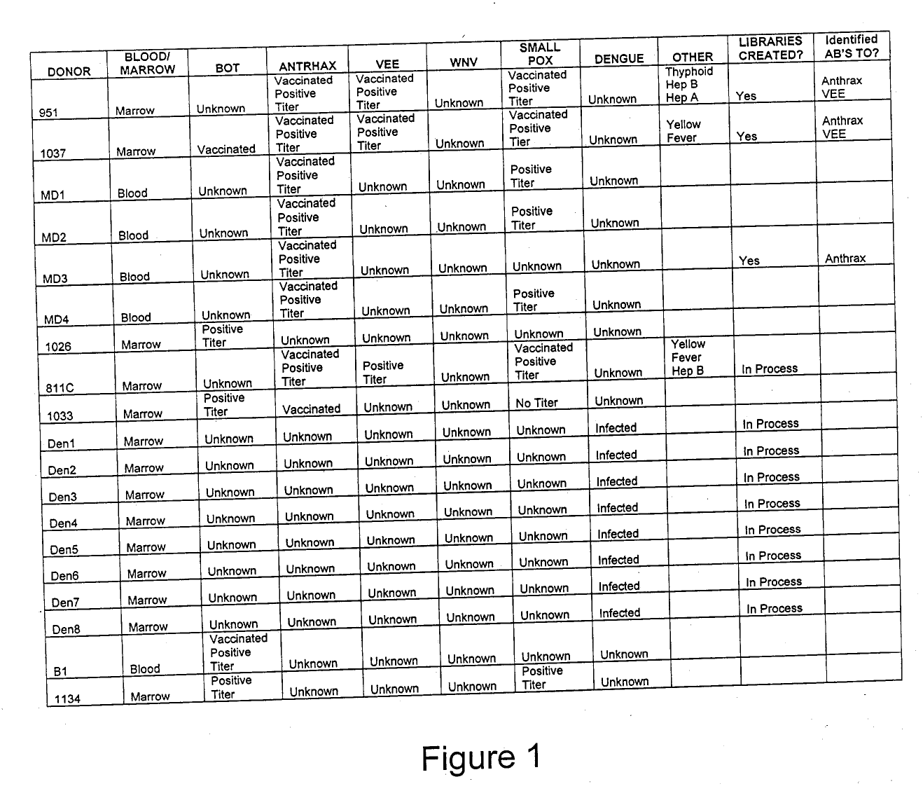

Generally, the first step in producing an antibody library in accordance with this disclosure involves collecting cells from an individual that is producing antibodies against one or more infective agents or antigens from infective agents. Typically, such an individual will have been exposed to the infective agent and/or antigen from an infective agent. In particularly useful embodiments, the individual has been exposed to a plurality of infective agents or antigens from infective agents that are strategically important with respect to biowarfare. Such materials include agents selected from the group consisting of anthrax, antigens from anthrax, botulinum, antigens from botulinum, smallpox, antigens from smallpox, Venezuelan equine encephalomyelitis virus (VEEV), antigens from VEEV, dengue, antigens from dengue, typhoid, antigens from typhoid, yellow fever, antigens from yellow fever, hepatitis, antigens from hepatitis, West Nile virus (WNV) and antigens from WNV. FIG. 1 is a table summarizing the exposure history of individuals suitable for use in preparing antibody libraries in accordance with preferred embodiments of the present disclosure. Cells from tissue that produce or contain antibodies are collected from the individual about 7 days after infection or immunization. Suitable tissues include blood and bone marrow.

Once the cells are collected, RNA is isolated therefrom using techniques known to those skilled in the art and a combinatorial antibody library is prepared. In general, techniques for preparing a combinatorial antibody library involve amplifying target sequences encoding antibodies or portions thereof, such as, for example the light and/or heavy chains using the isolated RNA of an antibody. Thus, for example, starting with a sample of antibody mRNA that is naturally diverse, first strand cDNA can be produced to provide a template. Conventional PCR or other amplification techniques can then be employed to generate the library.

Screening of the antibody library can be achieved using any known technique such as, for example, by panning against a desired viral antigen. See Rader and Barbas, Phage Display, A Laboratory Manual, Cold Spring Harbor Laboratory Press, Cold Spring Harbor, N.Y. (2000). Certain antigens have been cloned and can be produced recombinantly for use as immunogens. Neutralizing ability can be assessed in cellular assays that determine the ability of the antibody to block the binding of the virus with cellular receptors. Once antibodies having in vitro neutralizing ability are identified, they can be tested in vivo in animal models.

Antibodies identified in this manner advantageously provide an effective treatment for infection by an infective agent. Because the present antibodies are fully human antibodies, they are safe and easily tolerated. In addition, multiple doses can be given without rapidly raising an anti-idiotype response. Where full length antibodies are used, the higher avidity and larger size (compared to single chain antibodies) may be preferred because they provide greater residence time within the patient's system.

A particulary useful method for producing antibody libraries in accordance with this disclosure and identifying and characterizing antibodies in accordance with the present disclosure is as follows:

Libraries.

Three Fab libraries containing either lambda or kappa light chains and an IgG heavy chain fragment (Fd) were derived from each of two bone marrow samples (951 and 1037, and 1 blood sample (MD3) see FIG. 1) of active military donors immunized against a variety of infectious agents.

Libraries can undergo selection and screening against a variety of infective agents, such as anthrax, Venezuelan equine encephalitis and botulinum, West Nile virus, vaccinia virus, and dengue.

Library Creation.

Total RNA is obtained from bone marrow and blood samples using Tri-reagent BD (Molecular Research Center, Inc.) according to the manufacturer's instructions. Messenger RNA is obtained using Oligotex (Qiagen) spin columns per manufacturer's instructions. Phage libraries expressing antibody Fab fragments (kappa or lambda light chains complexed to the IgG heavy chain fragment (Fd) are constructed in plasmid vectors using the methods described in U.S. application Ser. No. 10/251,085 (the disclosure of which is incorporated herein in its entirety by this reference). Two Fab libraries are generated for each donor, one expressing kappa light chains and one expressing lambda light chains, and all utilizing gamma heavy chains.

Library Selection.

Phage bearing Fabs from all of the libraries used are panned through one to four rounds of enrichment against selected viral antigens and toxins. Panning is performed by first incubating a sufficient amount of recombinant antigen (usually 1-2 ug) in 50 μl of Solution A in several Immulon 2 HB microtiter wells overnight at 4° C. Solution A is phosphate buffered saline (PBS), pH 7.4, containing 0.08% boiled casein solution (BC). BC is PBS containing 0.5% casein, 0.01% thimerosal, and 0.005% phenol red. After removal of the antigen solution, wells are blocked for 1 hour at 37° C. with 250 μl of BC containing 1% Tween 20. Phage stocks are diluted into Solution D, consisting of BC with 0.025% Tween 20, and 50 μl are added to each well and incubated for 2 hours at 37° C. Wells are washed ten times with PBS containing 0.05% Tween 20, and then washed once for 2 minutes each with a progressively more acidic series of buffers (D'Mello et al., J Immunological Meth 247:191-203 (2001)): Tris-buffered saline (50 mM Tris-HCl, 150 mM NaCl) at pH 5.0, 4.0, and 3.0. Final elution is with 0.1 M glycine-HCl buffer, pH 2.2, 1 mg/ml bovine serum albumen (BSA). The eluent is neutralized with 2M Tris base and added to log phase ER2738 cells. Phage is produced by addition of helper phage (strain VCSM13) to infected bacteria. Individual colonies are generated by infecting susceptible bacteria with phage stock and plating.

Screening is done with supernatants containing Fab as a fusion protein with a portion of the phage gene III. After screening, positive candidates are sequenced and then subcloned to remove gene III prior to production of Fab for testing. Alternatively, DNA from each panned library can be subcloned to remove the gene III fusion region, and a combination epitope tag can be introduced, consisting of an influenza hemagglutinin epitope tag (HA) (Chen et al., Proc Nati Acad Sci USA 90:6508-12 (1993)) and six histidine amino acids (His tag) for use in subsequent detection and purification by anti-HA and Ni-NTA.

Library Screening.

For screening, Fab constructs reactive to the antigen of choice are identified by their ability to bind in an ELISA assay. 100 to 250 ng/well of recombinant antigen in Solution A is incubated overnight in Immulon microtiter dishes and blocked as described above. Screening can be performed in high-throughput by picking 1150 colonies using a Q-pix instrument, and performing ELISAs using a Tecan robot. Individual colonies are grown overnight in deep-well microtiter dishes in a Hi-Gro high-speed incubator shaker. Aliquots are removed and stored with 15% glycerol or 10% DMSO as stocks. After centrifugation of the deep-well dishes, supernatants containing Fab from these stocks are incubated in the wells coated with specific antigens and separately in wells coated with a control antigen such as casein or ovalbumin. Alkaline phosphatase labeled goat anti-human F(ab′)₂ antibody (Pierce) is used to detect Fab bound to antigen. Miniprep DNA (Qiagen) from positive candidates is sequenced by automated dye terminator sequencing (Retrogen, San Diego) in 96 well format across the light and heavy chains using stock primers for these vectors. Sequences are analyzed using DNAstar software to identify and classify unique candidates. From these data a panel of unique variant binders to each recombinant antigen used is determined, and classified into groups of closely related sequences.

Production and Purification of Fabs from Panels

Fab Purification.

For soluble Fab expression and purification, the gene III region is removed from unique positive candidates by subcloning. At this point it is also possible to insert an oligonucleotide that will encode a combination epitope tag consisting of an influenza virus hemagglutinin (HA) tag (Chen et al., Proc Natl Acad Sci USA 90:6508-12 (1993)) and six histidine residues (His tag) for detection and purification with anti-HA and/or Ni-NTA.

To purify sufficient Fab for ELISA based assays and in vitro neutralization tests in a higher throughput format, nickel-NTA column chromatography (Qiagen) is used. In this case, Fabs that have been subcloned (either before or after screening) to include a His tag are grown in 1 liter of SB to an OD₆₀₀ of 0.8 and induced with 1 mM isopropyl-β-D-thiogalactopyranoside (IPTG) for 3-4 hours at 30° C. to produce optimum amounts of Fab. To isolate Fab from the periplasmic space, cell pellets are resuspended in cold 1X PBS with added Complete Mini (Roche) protease inhibitor and are sonicated using a Sonics Vibra-cell VC750. Cellular debris is then pelleted and the supernatants are applied to Qiagen Ni-NTA columns. By using 16 of these columns, 75 μg of Fab per candidate was obtained in initial tests. By using a row of 12 columns per Fab in a single 96-well format, 8 Fabs can be purified, providing sufficient material for initial PRNT and ELISA assays. The epitope specificity tests require untagged Fab as well. These Fabs are purified on columns composed of goat anti-human F(ab′)₂ (Pierce) bound to Protein G or Protein A (Pharmacia) as described above in a 96 well format. Larger volumes of any desired Fabs can be purified by fast performance liquid chromatography (FPLC) (Pharmacia) on either the anti-human F(ab′)₂ column or on a nickel column. This method generally yields about 150-1000 pg of purified Fab/liter, though this varies from Fab to Fab.

Characterization of Purified Fabs.

Titration on Antigen.

Purified Fabs are titered against antigen in ELISA assays to compare the antigen-binding characteristics of Fabs within related groups established by sequencing.

Assays to Determine Epitope Specificity.

Epitope specificity can be determined by ELISA sandwich assays or by competition assays. Competition between Fab fused to gene III (fusion Fab, with or without phage attached) or a tag and purified Fab lacking gene III or a tag can be performed to assess epitope specificity. 50 μl of antigen at 4 μg/ml in PBS is incubated overnight at 4° C. in microtiter wells. After washing with PBS, wells are blocked with BC containing 1% Tween 20 in PBS at room temperature for 30 minutes. 50 μl PBS containing dilutions of one purified Fab are added to blocked wells and allowed to incubate at 37° C. for 1 hour. To this is added 50 μl of supernatant containing the second Fab as a fusion, and incubation proceeds for another hour at 37° C. The second Fab is detected with horse radish peroxidase-conjugated anti-M13 antibody (Pharmacia). Wells are developed with an HRP substrate buffer from Sigma, using 2,2′-azino-bis(3-ethylbenzthiazoline-6-sulfonic acid) tablets in a phosphate citrate buffer, pH 5.0. To use Fabs bearing the HA/His tag in this assay, the anti-M13 antibody used for detection above is replaced by either an alkaline phosphatase labeled anti-HA or labeled anti-His antibody detected with a PNPP assay.

Production and Purification of IgG from Fabs Identified as Neutralizing

Fabs are tested for their ability to neutralize the individual diseases using techniques known to those skilled in the art.

Conversion of Fabs to Full-Length IgG and Generation of Stable Cell Lines.

Fabs are subcloned in a two step process into a mammalian expression vector that creates a full-length IgG1 heavy chain. This vector utilizes a glutamine synthetase gene as a selectable marker, permitting growth of transfected cells in glutamine-free medium (Bebbington et al., Biotechnology 10:69-75. 1992). Vectors are transfected by electroporation using standard methods into the NS0 mouse myeloma cell line. Stable cell lines are selected in glutamine-free medium and are isolated by limiting dilution. Pooled transfections can also be performed with this vector in NSO or CHO-K1 cells in order to examine smaller quantities of IgG prior to selecting a stable cell line. DNA prepared from each clonal line is analyzed by restriction digestion to determine successful insertion of the vectored immunoglobulin. Western blot analysis of media from each clonal line is used to assess production of full-length IgG, and a quantitative ELISA assembly assay is performed by capturing light chains and detecting heavy chains with appropriate antibody.

For purification of IgG, transiently infected cells or stable cell lines expressing IgG candidates are grown in miniPerm bioreactors (Vivascience) or in hollow fiber bioreactors. Supernatants are purified by FPLC using a protein G or protein A column. Additional purification can be accomplished using a hydrophobic interaction column.

In Vitro and In Vivo Testing of IgG

IgG derived from Fabs can be tested in vitro and in vivo in assays specific for the individual diseases as described below.

The above techniques have been successfully used in the cases of anthrax and VEEV. The same libraries and/or libraries created from additional human donors can be panned against Dengue Virus, WNV, and Vaccinia Virus. The same techniques for converting Fabs to whole IgG and IgG purification can be used.

The present antibodies or antibody fragments may be used in conjunction with, or attached to other antibodies (or parts thereof) such as human or humanized monoclonal antibodies. These other antibodies may be catalytic antibodies and/or reactive with other markers (epitopes) characteristic for a disease against which the antibodies are directed or may have different specificities. The antibodies (or parts thereof) may be administered with such antibodies (or parts thereof) as separately administered compositions or as a single composition with the two agents linked by conventional chemical or by molecular biological methods. Additionally the diagnostic and therapeutic value of the antibodies may be augmented by labeling the antibodies with labels that produce a detectable signal (either in vitro or in vivo) or with a label having a therapeutic property.

The present antibodies or antibody fragments herein may typically be administered to a patient in a composition comprising a pharmaceutical carrier. A pharmaceutical carrier can be any compatible, non-toxic substance suitable for delivery of the monoclonal antibodies to the patient. Sterile water, alcohol, fats, waxes, and inert solids may be included in the carrier. Pharmaceutically accepted adjuvants (buffering agents, dispersing agent) may also be incorporated into the pharmaceutical composition. It should be understood that compositions can contain both entire antibodies and antibody fragments.

The antibody and/or fragment compositions may be administered to a patient in a variety of ways. Preferably, the pharmaceutical compositions may be administered parenterally, e.g., subcutaneously, intramuscularly, epidurally or intravenously. Thus, compositions for parenteral administration may include a solution of the antibody, antibody fragment, or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of antibody or antibody fragment in these formulations can vary widely, e.g., from less than about 0.5%, usually at or at least about 1% to as much as 15 or 20% by weight and will be selected primarily based on fluid volumes, viscosities, etc., in accordance with the particular mode of administration selected.

Actual methods for preparing parenterally administrable compositions and adjustments necessary for administration to subjects will be known or apparent to those skilled in the art and are described in more detail in, for example, Remington's Pharmaceutical Science, 17^{th} Ed., Mack Publishing Company, Easton, Pa. (1985), which is incorporated herein by reference.

### EXAMPLE 1

### Anthrax

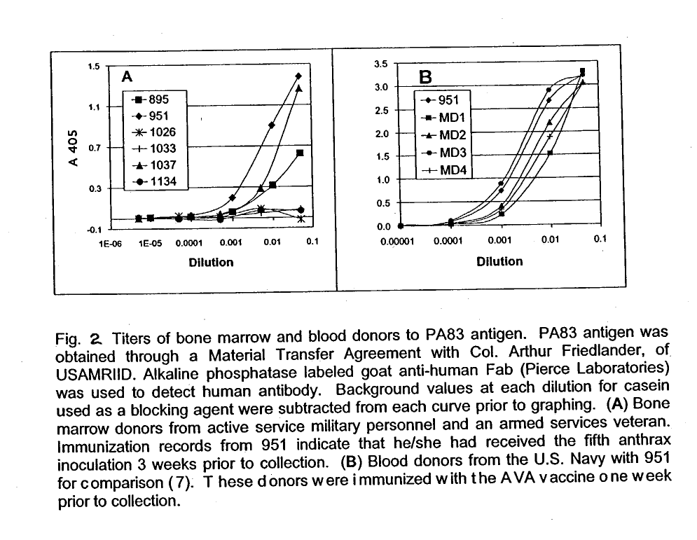

Phage libraries were developed from messenger RNA isolated from blood and bone marrow of active service military donors who had been vaccinated against anthrax. Blood samples were collected from military physician volunteer donors who received their AVA anthrax vaccine boost one week prior to collection. In addition, a commercial source supplied coded bone marrows with matched sera and some immunization records from active service military personnel. Several of the bone marrow donors and all of the blood donors had titer to anthrax antigen PA83 (FIG. 2). The bone marrow donor with the best titer against PA83 (951) had been immunized against anthrax three weeks prior to blood collection.

Total RNA was obtained from bone marrow samples 951 and 1037 and blood sample MD3 using Tri-reagent BD (Molecular Research Center, Inc.) according to the manufacturer's instructions. Messenger RNA was obtained using Oligotex (Qiagen) spin columns per manufacturer's instructions. Phage libraries expressing antibody Fab fragments (kappa or lambda light chains complexed to the variable and first constant regions of the heavy chain) were constructed in plasmid pAX243h vectors by proprietary methods as described in U.S. Provisional Application Nos. 60/287,355 and 60/323,455, the disclosures of which are incorporated herein in their entirety by this reference. Two Fab libraries were generated for each donor, one expressing kappa light chains, and one expressing lambda light chains, and all utilizing gamma heavy chains. Phage bearing Fabs from six libraries were panned through four rounds of enrichment against PA83. The 951 libraries were also separately panned through four rounds of enrichment against purified PA63, which was generated from PA83 as described by Miller et al. (Miller et al., 1999). To remove phage that bound to PA63 sites shared with PA83, soluble PA83 was first allowed to bind at 20 ug/ml to the phage for one hour at 37° C., after which the mixture was incubated with PA63 bound to microtiter plate wells.

Recombinant PA83 antigen was obtained from USAMRIID at Fort Detrick and was used in ELISA assays to identify anthrax-vaccinated individuals from the armed forces who have the highest titers against the PA83 antigen. RNA has been isolated from the bone marrow or blood of these individuals, and a Restriction Enzyme Digestion/Nested Oligonucleotide Extension Reaction/Single Primer Amplification (RED/NOER/SPA) was used to obtain combinatorial Fab libraries from this RNA. See FIG. 2.

RNA from the three highest titer individuals was used to construct libraries using the RED/NOER/SPA method of amplification. Two libraries, 951 and 1037, were from bone marrow donors received from Poietics (Menlo Park, Calif.). The third library, MD3, was from the blood of a vaccinated volunteer. The efficiency of the library ligations is shown in the following Table 1:

TABLE 1

| Library | Light Chain | Heavy Chain |
|---|---|---|
| 951 Kappa | 5.7 × 10⁹ | 2.17 × 10⁹ |
| 951 Lambda | 2.6 × 10⁹ | 1.86 × 10⁹ |
| 1037 Kappa | 3.07 × 10⁹ | 3.29 × 10⁹ |
| 1037 Lambda | 4.52 × 10⁹ | 1.07 × 10⁹ |
| MD3 Kappa | 6.65 × 10⁸ | 2.6 × 10⁸ |
| MD3 Lambda | 8.7 × 10⁸ | 4.2 × 10⁸ |

All of the libraries were panned against PA83, and the 951 libraries were panned against PA83, antigen was bound to wells and blocked prior to addition of phage bearing displayed Fab fragments. For PA63, the display phage were initially mixed with PA83 before being reacted with PA63 antigen bound to wells, in order to diminish the recovery of phage that reacted to antigens shared by both PA83 and PA63. PA63 was generated and purified from PA83 following the method described by Miller et al. (1999). The results for panning of two of the libraries and the panning against PA63 are shown in the following table.

Panning was performed, initially with the 951 and MD3 libraries against PA83, and with the 951 libraries on PA63. ER2738 cells were used, aside from the initial library transformations into XL1-Blue. Input, output, and some initial ELISA results for both panning rounds are shown in the following tables.

TABLE 2
Panning against PA83 and PA63

| | | | Input titer | Output titer | % ELISA |
|---|---|---|---|---|---|
| Library | Round | Antigen | (total) | (total) | positive |
| 951 K | 1 | PA83 | 5.9 × 10¹⁰ | 1.2 × 10⁵ | |
| | 2 | PA83 | 1.1 × 10¹⁰ | 3.5 × 10⁵ | 10% |
| | 3 | PA83 | 9.1 × 10¹⁰ | 1.2 × 10⁶ | 96% |
| | 4 | PA83 | 4.5 × 10¹⁰ | 1.0 × 10⁸ | 96% |
| 951 K | 1 | PA63 | 3.9 × 10¹⁰ | 1.9 × 10⁵ | |
| | 2 | PA63 | 5.0 × 10⁹ | 9.6 × 10⁵ | |
| | 3 | PA63 | 5.0 × 10¹⁰ | 7.6 × 10⁴ | |
| | 4 | PA63 | 2.5 × 10¹⁰ | 1.5 × 10⁵ | 15% |
| 951 L | 1 | PA83 | 8.8 × 10¹⁰ | 7.5 × 10⁴ | |
| | 2 | PA83 | 2.1 × 10¹⁰ | 7.3 × 10⁵ | 4% |
| | 3 | PA83 | 9.1 × 10¹⁰ | 1.7 × 10⁶ | 68% |
| | 4 | PA83 | 8.2 × 10¹⁰ | 1.6 × 10⁸ | 89% |
| 951 L | 1 | PA63 | 5.9 × 10¹⁰ | 2.3 × 10⁵ | |
| | 2 | PA63 | 5.6 × 10⁹ | 8.6 × 10⁵ | |
| | 3 | PA63 | 5.4 × 10¹⁰ | 4.8 × 10⁴ | |
| | 4 | PA63 | 3.3 × 10¹⁰ | 1.5 × 10⁵ | 15-30% |
| MD3 K | 1 | PA83 | 1.3 × 10¹¹ | 1.9 × 10⁴ | |
| | 2 | PA83 | 6.9 × 10¹⁰ | >10⁷ | 47% |
| | 3 | PA83 | 3.9 × 10¹⁰ | 3.5 × 10⁷ | 70% |
| | 4 | PA83 | 9.3 × 10¹⁰ | 1.1 × 10⁸ | 75-89% |
| MD3 L | 1 | PA83 | 4.8 × 10¹⁰ | 8.8 × 10⁴ | |
| | 2 | PA83 | 5.7 × 10¹⁰ | >10⁷ | 21% |
| | 3 | PA83 | 4.5 × 10¹⁰ | 8.3 × 10⁷ | 40% |
| | 4 | PA83 | 5.0 × 10¹⁰ | 1.7 × 10⁸ | 40% |

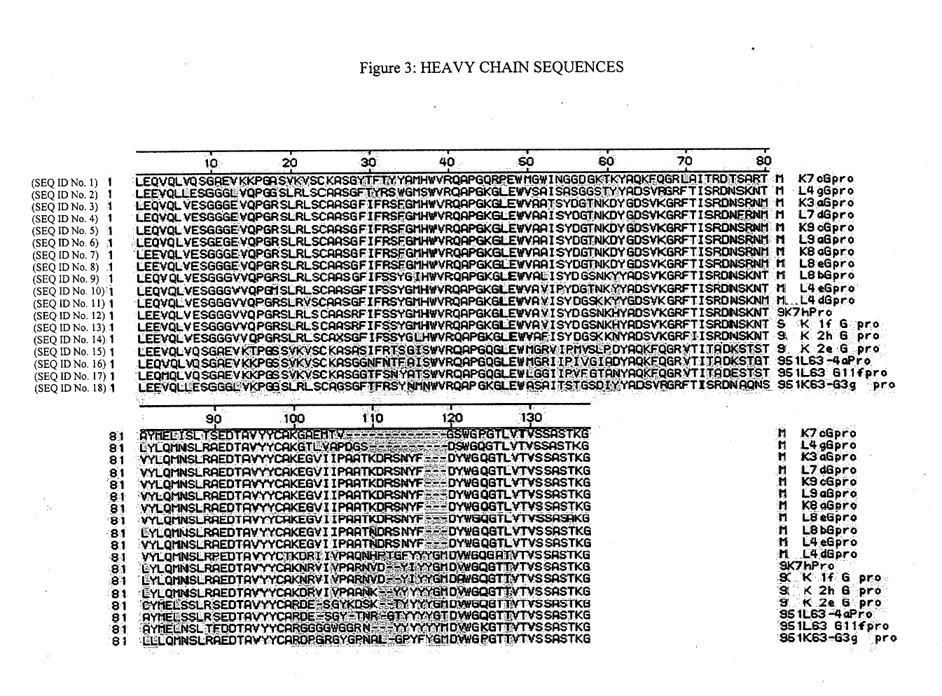

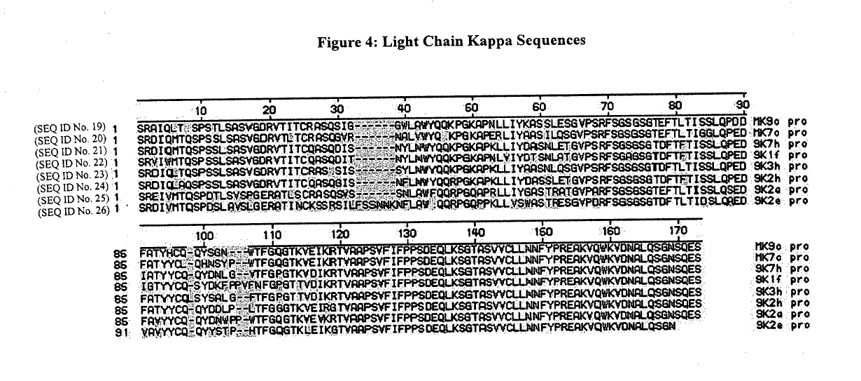

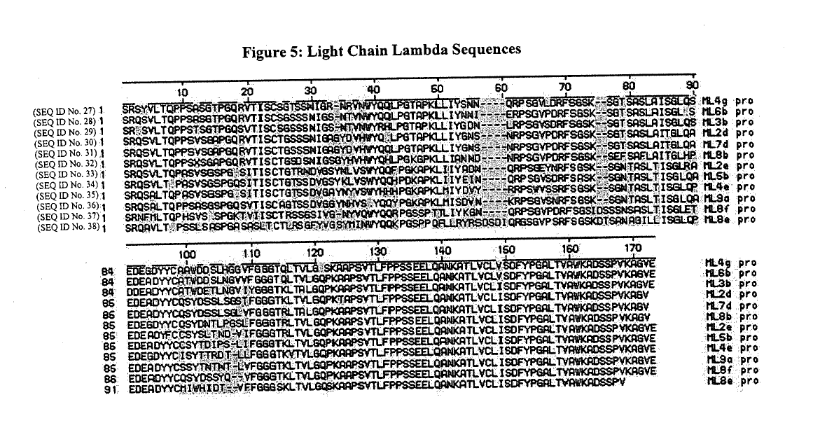

Enrichment is evident for all the PA83 panned libraries. Libraries panned against PA63 showed some candidates with very weak reactivity to PA83. These candidates were positive when tested against PA63. Sequence analysis of the VH and VK or VL regions of positive responders is indicated in FIGS. 3-5. Though certain sequences predominate, diversity can be demonstrated. The Fabs that were panned against PA63 with PA83 preabsorption appear to contain significantly different groups of sequences than those that were panned against PA83.

After panning, individual candidates from various panning rounds of all four PA83-panned libraries were screened for reactivity to PA83 by ELISA. In order to identify PA63 binding Fab fragments, 951 kappa and lambda library phage that had been panned against PA63 were first screened for binding to PA83, initially to eliminate PA83 binders from the screen. However, no candidates were found that bound PA83 well, indicating that the competition provided by incubating the phage initially with soluble PA83 was effective. A small percentage of clones in the fourth round panning of both the anti-PA63 libraries showed very weak ELISA reactivity after several hours of incubation in substrate. These clones were screened against PA63 resulting in a much stronger signal. The weak reactivity to PA83 may be due to cross-reactivity with PA83, or may reflect a small amount of PA63 in the PA83 preparation, which might have resulted from protease cleavage of PA83 at the furin protease sensitive site (Klimpel et al., 1992) during purification or storage.

Over 144 individual candidates with strong PA83 or PA63 binding activity selected from the six different panned libraries were sequenced and a panel of all variant candidates was identified. This included thirty-one unique PA83 binders and six unique PA63 binders. Twenty-five of the unique PA83 binders were all derived from variable heavy chain (VH) locus 3-30/3-30.5. Among the heavy chains of the PA63 binders, two related sequences were predominantly seen; these were dissimilar to the PA83 sequences. Because a single mutation can dramatically alter the affinity of an antibody, candidates were considered unique if they had one amino acid difference in either their heavy or light chains as compared to other candidates.

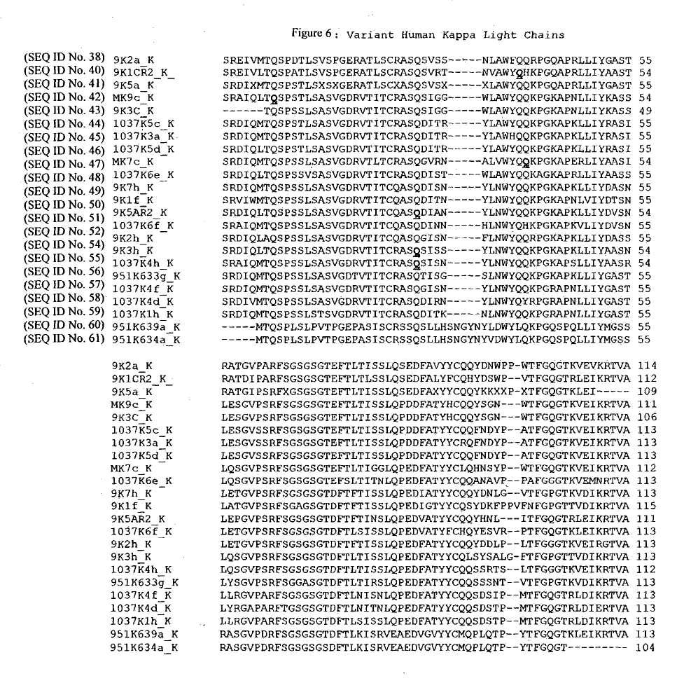

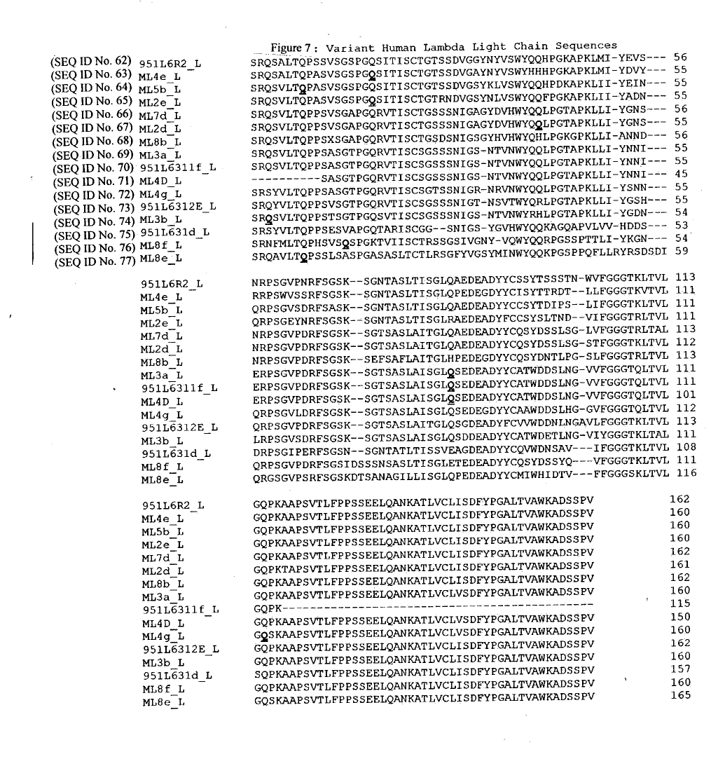

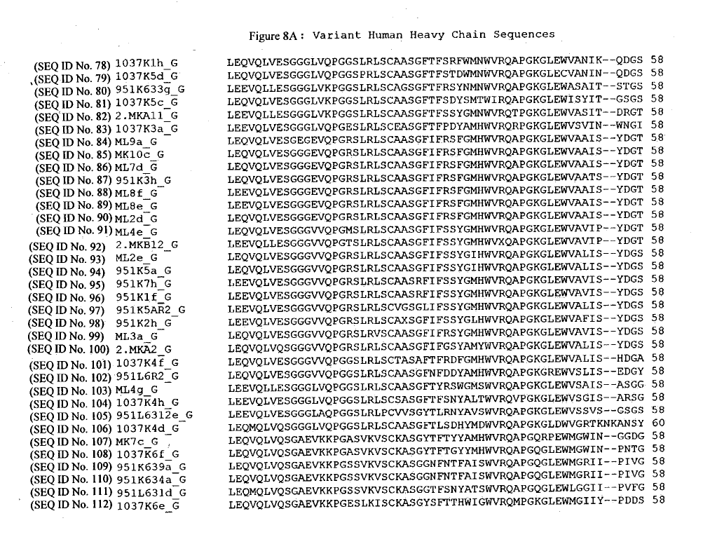

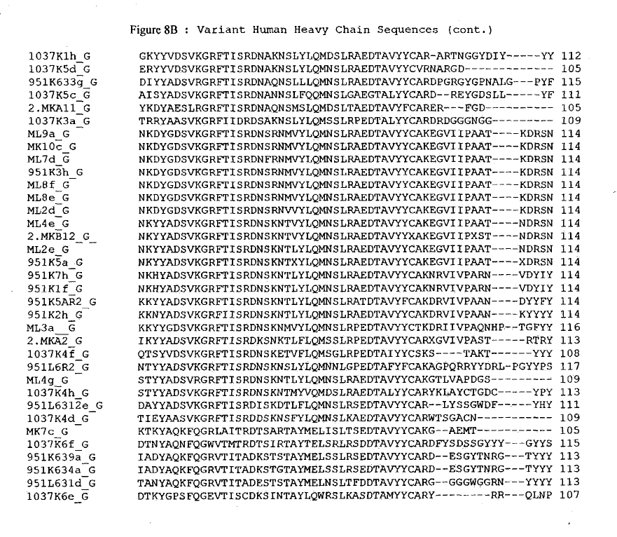

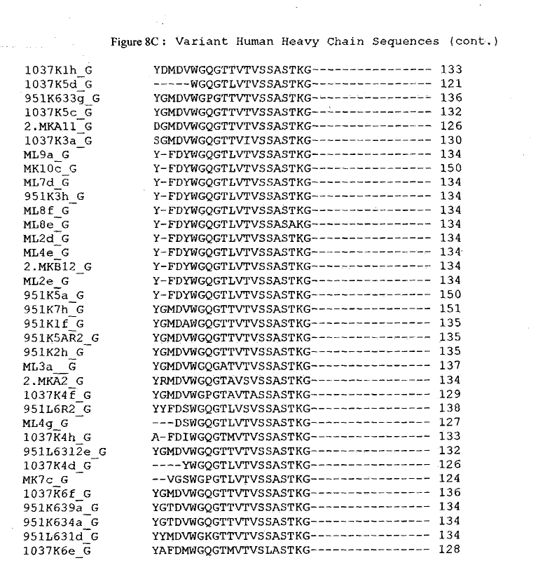

Additional antibody sequences to the anthrax proteins PA83 and PA63 are presented in FIGS. 6-8C. For the human kappa light chain variable sequences shown in FIG. 6, the first two amino acids, S (serine) and R (argentine) are derived from the Xba I (TCTAGA) site used in cloning. Amino acid number three in the figure corresponds to amino acid number one for human kappa light chains in the Kabat numbering system (Sequences of Proteins of Immunological Interest, Kabat et al., 1991). The last four amino acids (RTVA) indicated for most of the sequences corresponds to the first four amino acids of the human kappa light chain constant regions, numbered 108-111 in the Kabat numbering system. Two sequences shown do not quite extend to the beginning of the constant region. Because the variable region includes length polymorphisms, the actual number of amino acids in each sequence may be larger or smaller than 113 (the two initial amino acids, plus 111). For the human lambda light chain variable sequences shown in FIG. 7, the first two amino acids, S (serine) and R (argentine) are derived from the Xba I (TCTAGA) site used in cloning. Amino acid number three in the figure corresponds to amino acid number one for human lambda light chains in the Kabat numbering system. The last amino acid indicated for each sequence corresponds to amino acid 155 of the human lambda light chain constant regions in the Kabat numbering system. Because the variable region includes length polymorphisms, the actual number of amino acids in each sequence may be larger or smaller than 157. For the human gamma heavy chain variable sequences shown in FIGS. 8A-C, the first two amino acids, L (leucine) and E (glutamate) are derived from the Xho I (CTCGAG) site used in cloning. Amino acid number three in the figure corresponds to amino acid number one for human gamma heavy chains in the Kabat numbering system. The last amino acid indicated for each sequence corresponds to amino acid 118 of the human gamma heavy chain constant regions in the Kabat numbering system. Because the variable region includes length polymorphisms, the actual number of amino acids in each sequence may be larger than 120.

Panning against EF and LF, which are also present in small amounts in the AVA vaccine used to immunize military personnel, is performed with the present libraries. Additional panning against PA63 can be performed with the other libraries. Biacore assays are done to assess affinity of the different antibodies. Competition experiments are performed to identify groups of antibodies that share the same epitope binding characteristics. Candidates are assessed for their ability to block the binding of PA with either receptor, EF or LF in cellular assays. The best candidates are then tested for their ability to block toxicity in vivo in animal models, either using PA, EF and LF or actual anthrax infection. Candidates are optionally converted to full length human antibodies one or more of these tests.

To generate purified Fab for additional testing, candidates from this panel underwent a subcloning step to remove gene III from the heavy chain portion of the Fab fragment. Fab was then purified from two to four liters of culture by fast performance liquid chromatography (FPLC) using a goat anti-human Fab column. Neutralization assays using the purified Fab were performed using a mouse macrophage cell line, J774A.1, after the manner of Little et al. (Little et al., 1990). Conditions were established for using the Cytotox96 detection kit (Promega) to assay lactate dehydrogenase (LDH) released by cell death in response to toxin action. J774A.1 cells were plated overnight at 14,000 cells/well in 96 well dishes. 4-8 wells were assayed for each point. Fabs were used at 50 nM. Toxin was generated as follows: PA83 was added at 400 ng/ml (4.6 nM), with LF at 40 ng/ml. After incubation at 37° C. for 4 hours, wells were examined microscopically and then media was removed and centrifuged to pellet unattached cells.

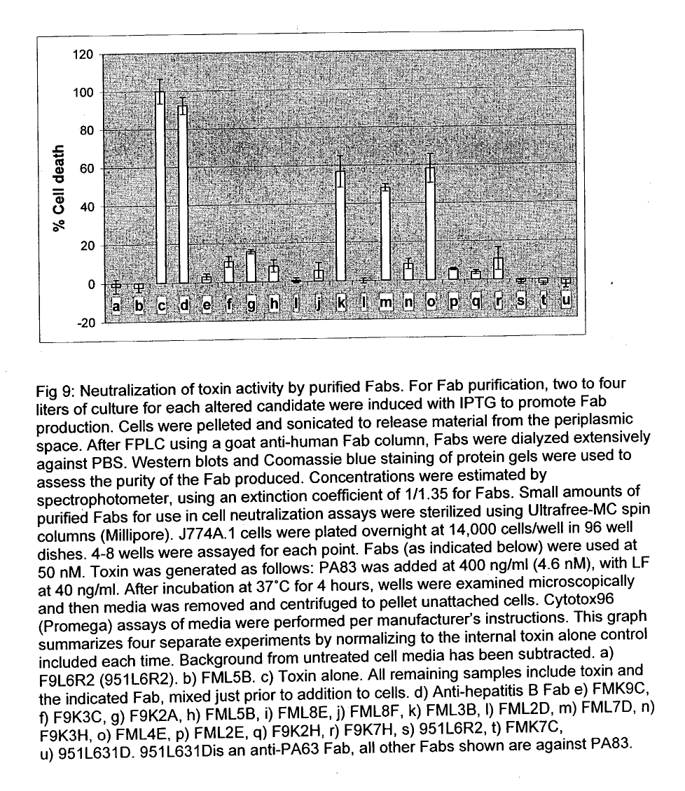

Results of a number of neutralization assays are summarized in FIG. 9. As can be seen, fourteen of the seventeen anti-PA Fabs (samples e-u) tested are able to neutralize the effects of the anthrax toxin with greater than 80% viability. Five Fabs neutralize fully at this concentration and in this time frame. Samples (a) and (b) are two of the Fabs without the addition of toxin; these demonstrate that cell death is not caused in this time period by endotoxin in the purified samples. Sample (c) shows the effect of toxin alone. Sample (d) contains an irrelevant Fab that does not protect cells significantly from the action of the anthrax toxin.

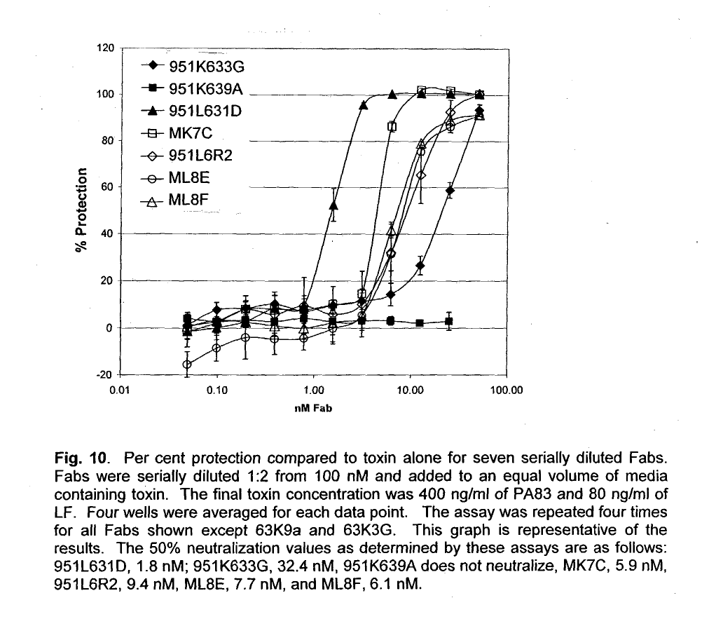

Selected Fabs were titrated to determine their 50% protection values in vitro (FIG. 10). Fabs were serially diluted and aliquots were added to media containing toxin. In these experiments, PA was at a final concentration of 400 ng/ml, and LF at 80 ng/ml. These aliquots were then added to cells in quadruplicate and incubated at 37° C. for 4 hours. Cytotoxicity was assessed visually and was quantitatively measured with the Cytotox96 assay as described. The anti-PA83 Fabs shown here all have 50% neutralization values that are close to equimolar with the concentration of PA83 used in the assay. The anti-PA63 Fab 951 L631 D, however, has a 50% neutralization value that is about 5-7 fold lower than these; in other words, one molecule of 951L631D neutralizes many molecules of PA83. PA83 is cleaved and converted by the J774A.1 cells in this experiment to heptameric pores. The most probable explanation for the ability of 951 L631 D to neutralize substoichiometric amounts of PA83 is that it is acting at the level of the heptameric pore, and is effectively neutralizing up to seven PA83 molecules at once. 951 L631 D and MK7C have recently been tested in vivo. Two rats receiving 40 μg of PA83 and 8 ug of LF in 200 ul total volume of PBS died in 60 and 71 minutes. Two rats receiving the same quantities of toxin and 310 pg of 951L631D survived for 25 hours, at which time they were sacrificed. At about 3-5 hours, these rats showed some symptoms of illness, such as lethargy and a slight panting, but at 16 hours this had disappeared in one rat, while the other remained lethargic but had normal breathing. By 25 hours both rats appeared normal as compared to the PBS injected control rat. 951L631D therefore appears capable of protecting rats against anthrax intoxication in vivo. MK7C has been tested at 300 pg with toxins in one rat which survived without showing any symptoms.

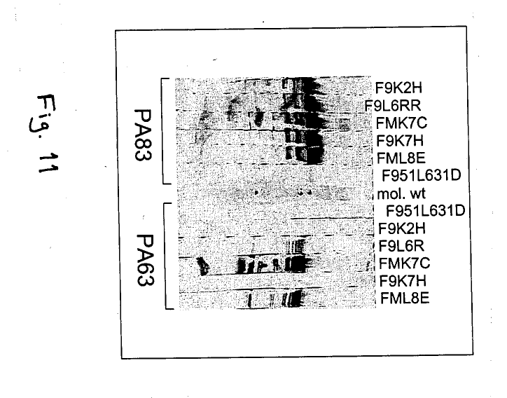

Fabs generated from 9K2H, 9L6R2, MK7C, 9K7H, ML8E, and 951L631D were tested for their ability to react with linear epitopes. PA83 and PA63 were run under denaturing (but non-reduced) conditions in an SDS-PAGE gel and transferred to nitrocellulose filters by Western blotting. Strips cut from the blots containing either PA63 or PA83 were hybridized to each of these purified antibodies overnight at the same concentrations. Bound antibody was reacted with alkaline phosphatase conjugated goat anti-human F(ab′)₂ (Pierce), and the results are shown in FIG. 11.

All of the five anti-PA83 Fabs tested appear to bind to linear epitopes on PA83 (FIG. 12). The anti-PA63 antibody, in contrast does not bind to denatured PA63, and shows what appears to be faint, presumably non-specific binding to PA83. 9K2H and 9K7H show no binding to denatured PA63, whereas MK7C and ML8E bind strongly, with 9L6R2 showing weak binding.

The ability of some of these Fabs to bind to PA83 and PA63 was further analyzed quantitatively by performing a Fab titration against antigen in an ELISA format. PA83 and PA63 were purchased from List Laboratories and resuspended in water or 50% glycerol, respectively, per instructions. The graph below shows the titration of four Fab fragments against PA83 or PA63. Closed symbols represent reactivity to PA83, open symbols to PA63. The three antibodies generated against PA83 all show strong reactivity to PA83 as expected. However, only MK7C reacts equally well to PA63, indicating that the cleavage of PA83 to PA63 alters or removes the epitopes seen by 951 L6R2 and ML8E. 951 L631 D does not react with PA83, but does react with PA63, though at a much lower saturation level than seen with MK7C reacting with PA63. These assays were performed in the same plate at the same time in duplicate. This suggests that only a portion of the PA63 present on the plate presents the proper epitope for binding to 951L631D, as might be expected if 951L63-1D were to react with an epitope found only in the heptameric pore structure, which may be underrepresented on the plate.

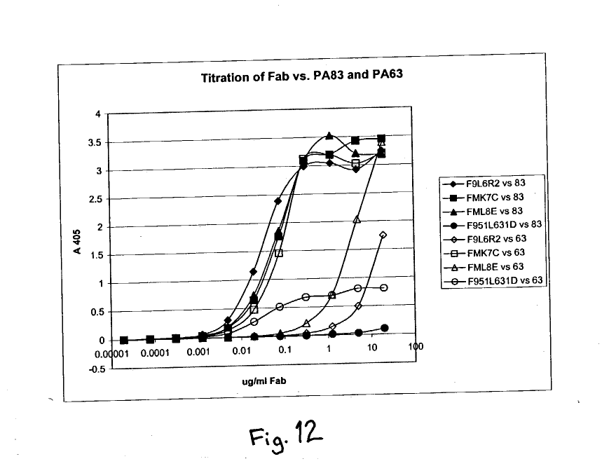

In FIG. 13, a his tagged version of Fab FML8E was generated and used in competition with other untagged Fabs to assess epitope specificity. Fabs F9K2H, F9K7H, and FML8F all compete similarly to self-competition with FML8E, suggesting that these Fabs recognize the same epitopes, or epitopes in close proximity to that seen by FML8E. F951L6R2 competes, but not as well, suggesting that this epitope is not the same, though it may be close enough to cause competition. FMK7C is very ineffectual in competition, indicating that it probably binds at a distant site. Interestingly, cleavage to PA63 abolishes binding by F9K2H and F9K7H, as shown in the Western blot figure above, while binding on the Western is still evident for FML8E and F951 L6R2, and some reactivity is also seen at high concentrations in the ELISA titration above. This suggests that the epitopes for binding F9K2H/F9K7H are not the same as for FML8E/FML8F, F951 L6R2, or FMK7C. Fabs indicated were serially diluted 1:4 and bound for one hour at 37° C. to microtiter wells that had been coated overnight with 200 ng of PA83. His-tagged FML8E was then added without washing at 5 ug/ml and allowed to react for 2 hours, after which plates were washed and reacted with alkaline phosphatase conjugated anti-His for a PNPP assay. Note that FML8E and FML8F have similar heavy chains, but different light chains. F9K2H and F9K7H are related to each other and use the same heavy chain germline locus as FML8E, but have quite different CDR regions from ML8E. F951 L631 D and FMK7C are from different heavy chain germline loci.

This disclosure demonstrates for the first time that human anti-anthrax toxin antibodies which possess a high affinity and are potently neutralizing in vitro, can be isolated from AVA immunized donors. Little et al. (1990) identified a panel of murine monoclonal antibodies against the anthrax toxin lethal factor. Evaluation of in vitro versus in vivo protection suggests that the degree of protection in vitro may correlate with protection in vivo, except for rare cases. Fifteen neutralizing antibodies have been identified from the nineteen examined, some of which neutralize fully at low concentrations. It is anticipated, therefore, that some of these antibodies will be protective in vivo. The data further suggests that the AVA vaccine is effective in protecting humans against anthrax exposure.

Both anti-PA83 and anti-PA63 activities in combination have potential for in vivo therapeutic purposes. Anti-PA83 would limit the number of PA83 molecules binding to cellular receptors. Those PA83 molecules that were not destroyed and did form heptameric pores would then be neutralized by anti-PA63 activity, providing potent protection against the lethal effects of an anthrax infection. The combination of the two antibodies could provide immediate protection against the formation of new functional pore structures either at the onset or during the course of an infection.

The use of these two antibodies could provide additional passive protection to personnel, vaccinated or unvaccinated, that might be exposed to a suspected anthrax release. The availability of a therapeutic that could protect in the face of disease would help to alleviate public anxiety about anthrax. In addition, such a therapeutic agent might make the deliberate release of anthrax less successful as an act of bioterrorism, and may therefore decrease the likelihood of such attacks.

### EXAMPLE 2

### Venezuelan Equine Encephalitis Virus

Human Anti-VEEV Abs

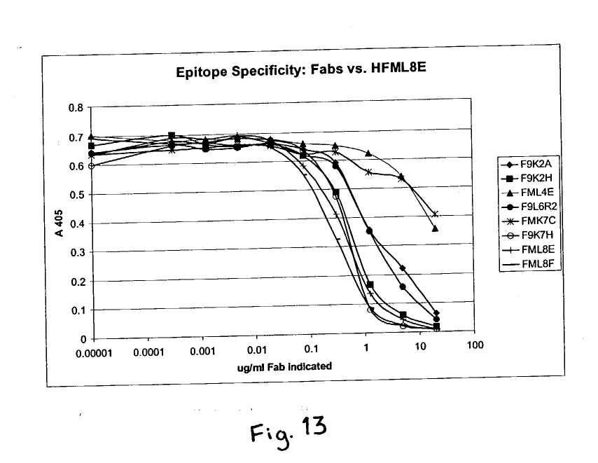

The donor serums described above in connection with Example 1 were tested against TC-83 antigen using a standard ELISA assay (FIG. 14). Donors 1037, 811 and 951 had significant serum reactivity against TC-83. This indicated a high probability of obtaining anti-VEEV Fabs from antibody libraries made from the corresponding donor bone marrow. The IgG-kappa and IgG-lambda libraries for both 1037 and 951 (4 libraries in total) had previously been constructed for the anthrax example as described above. These phage-display antibody libraries were then panned through 4 rounds on TC-83 antigen. Results from this experiment are shown below:

| Initial Library Sizes: | | |
|---|---|---|
| 951 K | 5.7 × 10⁹ | |
| 951 L | 2.6 × 10⁹ | |
| 1037 K | 3.1 × 10⁹ | |
| 1037 L | 4.5 × 10⁹ | |
| | Input | Output |
| Round 1 Panning: | | |
| 951 K | 5.6 × 10¹¹ | 6.0 × 10⁴ |
| 951 L | 2.6 × 10¹¹ | 1.8 × 10⁵ |
| 1037 K | 4.8 × 10¹¹ | 4.0 × 10⁴ |
| 1037 L | 3.2 × 10¹¹ | 1.0 × 10⁵ |
| Round 2 Panning: | | |
| 951 K | 1.2 × 10¹³ | 1.2 × 10⁷ |
| 951 L | 3.0 × 10¹³ | 1.0 × 10⁶ |
| 1037 K | 3.8 × 10¹³ | 4.4 × 10⁷ |
| 1037 L | 2.2 × 10¹³ | 4.8 × 10⁶ |
| Round 3 Panning: | | |
| 951 K | 5.0 × 10¹³ | 3.1 × 10⁸ |
| 951 L | 7.8 × 10¹³ | 4.0 × 10⁷ |
| 1037 K | 8.0 × 10¹³ | 6.1 × 10⁸ |
| 1037 L | 8.8 × 10¹³ | 2.1 × 10⁸ |
| Round 4 Panning: | | |
| 951 K | 1.0 × 10¹⁴ | 8.0 × 10⁹ |
| 951 L | 4.4 × 10¹³ | 2.0 × 10⁹ |
| 1037 K | 1.5 × 10¹⁴ | 1.0 × 10¹⁰ |
| 1037 L | 3.8 × 10¹³ | 4.0 × 10⁹ |

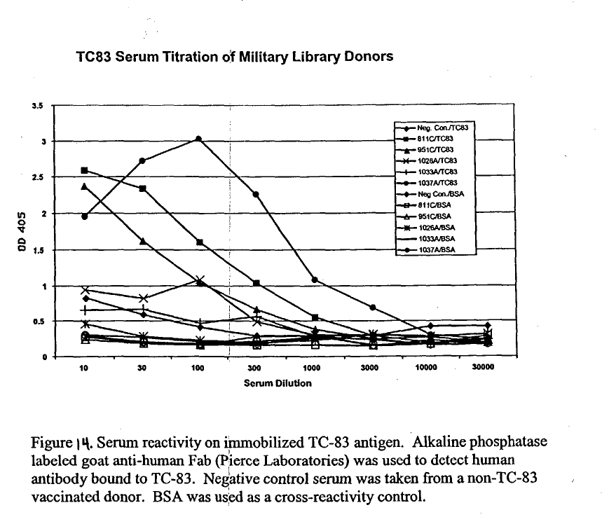

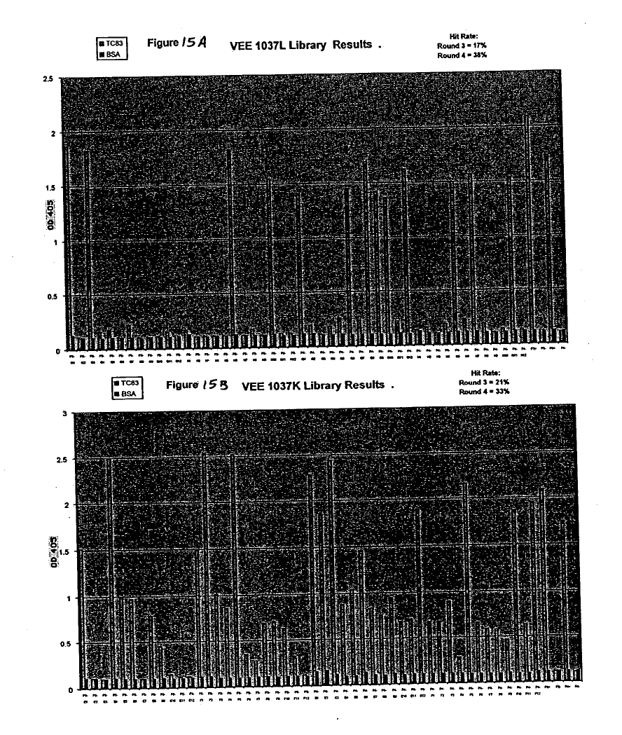

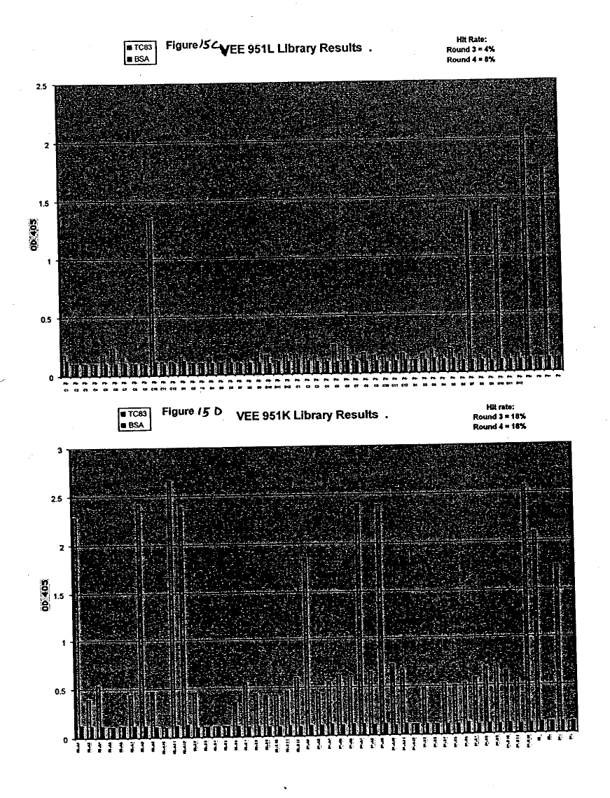

A panel of Fab clones from panning rounds 3 and 4 from all four libraries (951 K. 951L, 1037K and 1037L) was screened for binding to immobilized TC-83 by ELISA using a Tecan robotic platform in a high throughput format. As seen in FIGS. 15A-D, Fabs with significant binding to TC-83 (as detected using alkaline phosphatase conjugated anti-human Fab) were obtained in all 4 libraries. Fab clones were screened in comparison to positive control Hy4-26A (humanized variant of 3B4C-4) and a negative control anti-tetanus toxoid Fab which are the next to last and last samples respectively on each graph in FIG. 15. Three Fabs with the highest ELISA signals from each of the four libraries were selected for further analysis. DNA was prepared for each clone and then submitted for sequence analysis. The sequencing results showed that all three 951K clones were identical. Additionally, 10 of the 12 clones had the same variable heavy chain region (VH) but the majority of those Fabs had different light chain sequences. In all, there are 10 unique clones in 3 separate heavy chain (HC) groupings.

Four human Fab clones were chosen for further analysis. The selected Fabs represented all three distinct HC classes identified.

| | VH classification | |
|---|---|---|
| Clone | (generalized grouping) | LC |
| P3F2 | #1 | kappa |
| P3F5 | #2 | kappa |
| P3H6 | #3 | kappa |
| P3G1 | #1 | lambda |

All of the Fabs were purified from bacterial periplasmic preps using an anti-human F(ab′)₂ column on an FPLC. Because the P3H6 Fab had very low yield it was not pursued further.

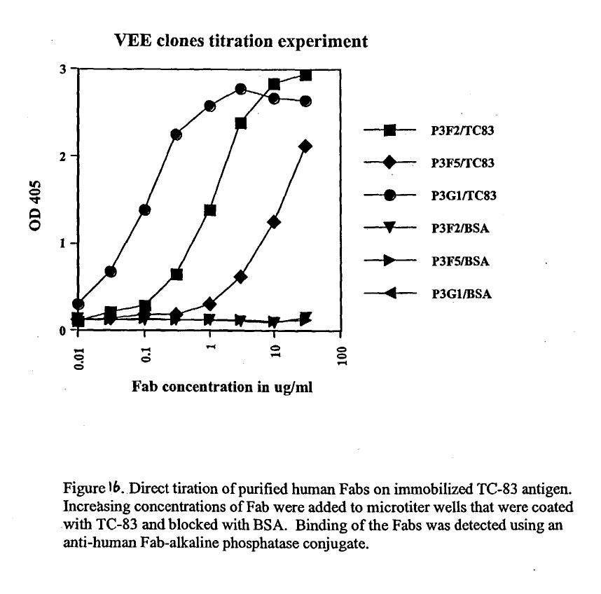

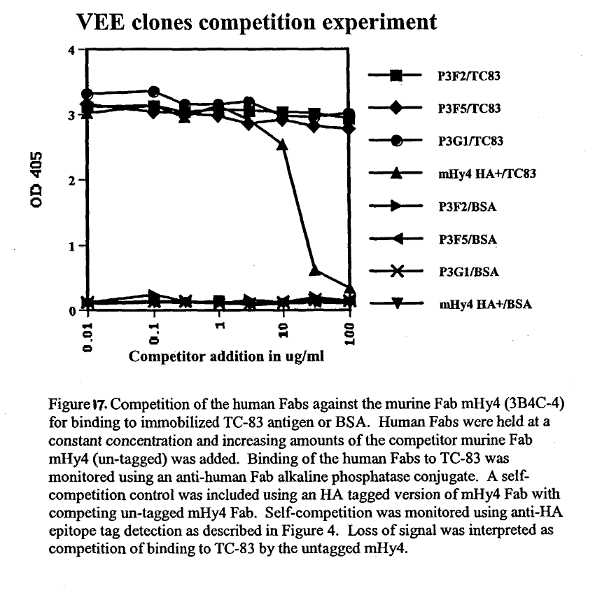

FIG. 16 shows the binding activities of the three human anti-VEEV Fabs in a titration ELISA assay against TC-83. The purified anti-VEEV Fabs were also tested in a competition ELISA experiment, using the mHy4 Fab as the competitor. The results from this experiment are shown in the FIG. 17 and demonstrate the three VEEV Fabs do not compete for the same epitope (E2^{c}) as the mHy4 Fab.

The human Fabs were not competitive for the E2^{c} epitope, but they may bind to other neutralizing epitopes on VEEV. To test that, an aliquot of each purified VEEV Fab was sent to collaborators at the CDC for use in a cell-based VEEV neutralization assay. The results from two separate experiments showed that P3F5 had very good neutralization capacity, similar to that seen with the positive control 3B4C-4. P3G1 also showed significant neutralization, while the P3F2 Fab had no apparent effect in the neutralization tests.

TABLE 3

| Fab/Ab | Sample type | Crosslinked? | 70% PRNT |
|---|---|---|---|
| 3B4C-4mAb | positive contol | no (already bivalent) | 25 ng/ml |
| TT | negative control | yes | >2500 ng/ml |
| P3F2 | Test | yes | >10,000 ng/ml |
| P3F5 | test | yes | 19.5 ng/ml |
| P3F5 | test | no | <78 ng/ml |
| P3G1 | test | yes | 156 ng/ml |
| P3G1 | test | no | 156 ng/ml |

Table 3 reports the results of in vitro neutralization assay for VEEV. The titer of Ab or Fab required to give 70% reduction of VEE viral plaques in Vero cells is reported. The murine Ab 3B4C-4 (as whole IgG) was used as a positive control. Previously, bivalent antibody has been shown to neutralize virus more effectively, therefore anti-Fab cross-linking Ab was added to some wells (non-optimized concentration). A non-binding negative control Fab did not show neutralization at any concentration tested. Samples P3F5 showed activity near that of the murine 3B4C-4.

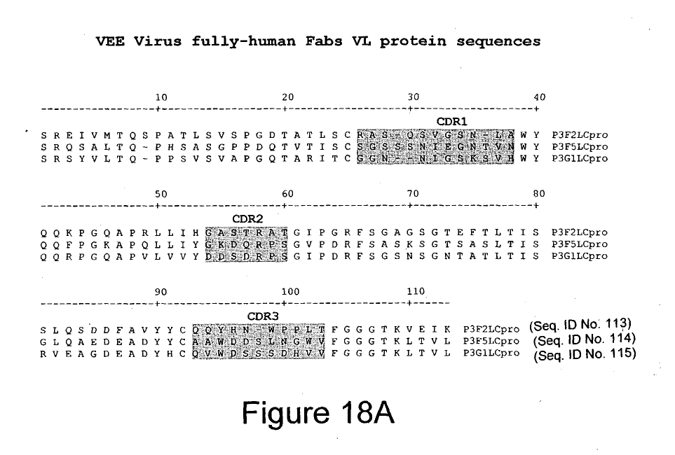

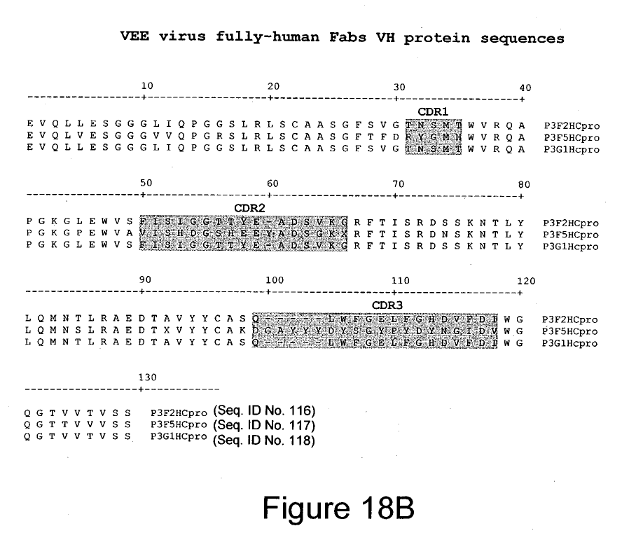

These preliminary results demonstrate that a fully human neutralizing anti-VEEV antibodies had been isolated. FIGS. 18A and 18B show the sequences for fully-human Fabs produced in accordance with this disclosure that neutralize VEEV. These existing human anti-VEEV Fabs can be converted to whole IgG as described above and purified for further characterization.

Test epitope specificity of the antibody for VEEV (Roehrig, et al Virology (1982) 118, pp269-278; and Roehrig and Mathews, Virology (1985) 142, pp 347-356).

Western Blot is run to see which of the TC-83 viral proteins is recognized by the Fabs. For Fabs that do not react by Western Blot, because a conformational rather than a linear epitope is recognized, native E1 and E2 envelope glycoproteins can be purified from viral lysate for ELISA or Radiolabeled immunoprecipitation assays as described previously.

Identification of the reactive epitope on the viral protein can be mapped using a competition ELISA with representative monoclonal antibodies for each binding group as listed below in Table 2. Microtiter wells coated with whole virus are incubated with an amount of the representative Ab that gives approximately 80% maximal binding. Wells also contain increasing amounts of the test Fab. Binding of the representative Ab to virus is monitored using an anti-mouse IgG Fc specific —Alkaline Phosphatase conjugate. Loss of binding is interpreted as competative binding by the test human Fab, indicating epitope specificity or spatial arrangement.

TABLE 4

| Representative Antibody | Epitope |
|---|---|
| 5B4D-6 | gp56^{a} |
| 2A4B-12 | gp56^{b} |
| 3B4C-4 | gp56^{c} |
| 1A6C-3 | gp56^{d} |
| 1A3A-5 | gp56^{e} |
| 1A4D-1 | gp56^{f} |
| 1A3A-9 | gp56^{g} |
| 1A3B-7 | gp56^{h} |
| 3B2D-5 | gp50^{a} |
| 3B2A-9 | gp50^{b} |
| 5B6A-6 | gp50^{c} |
| 3A5B-1 | gp50^{d} |

The representative Abs (from John Roehrig at the CDC, Ft. Collins, Colo.) can be obtained from ascitic fluid following a 50% ammonium sulfate precipitation and chromatography over a protein G column. Alternatively, Abs can be purified from the conditioned media of their hybridoma cell lines grown in Ig free media.

Test viral strain cross reactivity (Roehrig et al., J. Clin. Microbiology (1997) 35, pp1887-1890: and Roehrig et al., Virology (1982) 118, pp269-278).

VEEV is composed of six subtypes (1-6) with subtype 1 having five variants (1AB, 1C, 1D, 1E, and 1F). Virus strains from each subtype is tested by ELISA or indirect fluorescent antibody assay (IFA) as described previously for reactivity with each candidate Fab. Prototype viruses useful in these analysis are listed below in Table 5.

TABLE 5

| Strain | Subtype |
|---|---|
| TC-83 | 1AB |
| Trinidad Donkey (TRD) | 1AB |
| P676 | 1C |
| 3880 | 1D |
| Mena 2 | 1E |
| 78V-3531 | 1F |
| Everglades (Fe3-7c) | 2 |
| Mucambo (BeAn 8) | 3 |
| Pixuna (BeAr 35645) | 4 |
| Cabassou (CaAr 508) | 5 |
| Ag80-663 | 6 |
| Western Equine Encephalitis (WEE) | |
| (McMillan) | |
| Eastern Equine Encephalitis (EEE) (82V- | |
| 2137) | |
| St. Louis Encephalitis (SLE) (MSI-7) | |

Viruses from stocks maintained at the Division of Vector Borne Viral Diseases, Centers for Disease Control, Fort Collins, Colo., can be grown in BHK21 cells.

Perform In Vitro Neutralization Test with Whole IgG.

Neutralization tests are done using 50-100PFU/test in Vero cells, with 70% endpoints recorded as described previously (Roehrig et. al., 1982).

Test Ability of Abs to Protect Mice from Viral Challenge.

Known quantities of purified IgG diluted in PBS are inoculated i.v. via a tail vein into young mice, such as 3 week old NIH Swiss mice. Twenty-four hours later, mice are challenged i.p. with VEEV diluted in cell culture media. Controls receive PBS i.v. and either virus or virus diluent. An additional control group includes murine Ab 1A4A-1 or 3B4C-4 previously shown to provide protection. Mice are observed for 2 weeks. Heparinized plasma specimens from inoculated mice are obtained by bleeding from the reto-ocular venous plexus.

Isolation of Additional Anti-VEEV Fabs

An extended panel of human Fabs directed against TC-83 antigen is generated.

Additional ELISA screens of >1000 individual Fab clones from 1037 and 951 libraries which have already been panned on TC-83 are performed. This supplements the original screen of 190 Fab clones from those panned libraries. In addition, new phage display antibody libraries are constructed from the RNA of a donor (811) previously shown to have titer against TC-83. The newly constructed 811 libraries are panned on immobilized TC-83. Unique Fabs are characterized as described above for their ability to provide neutralization in vitro and protection in animal models against lethal viral challenge.

### EXAMPLE 3

### Botulinum

By applying the previously described library creation and panning technologies, antibodies that bind many of the different botulinum toxin serotypes are isolated and produced in large quantities. As with the neutralizing antibodies described above for anthrax and VEEV, these fully human antibodies against botulinum neurotoxins are suitable for immunoprophylaxis or as immunotherapeutics.

### EXAMPLE 4

### Dengue Virus

Human full-length neutralizing antibodies would be particularly useful as logical and natural anti-toxins or anti-infectives as they have already been proven to be safe and well tolerated for other therapeutic purposes. Neutralizing antibodies, either raised by vaccination in animals or passively administered to a variety of animal hosts, have been shown in some instances to provide protection against dengue. However, there are indications that infection with dengue in humans is potentiated by vaccination, and reports that antibodies against specific dengue antigens can themselves cause hemorrhage through cross-reaction with common epitopes on clotting and integrin/adhesin proteins (Falconar, 1997).

Generation of 16 antibody libraries from blood or bone marrow samples of 8 human donors infected or vaccinated with different serotypes of dengue virus are created. Two libraries are generated from each donor, one utilizing kappa light chains, and the other utilizing lambda light chains. The 8 donors include 4 donors singly infected or vaccinated with each of the four serotypes of dengue, two libraries from individuals infected with multiple dengue serotypes, and two libraries from individuals who have received the tetravalent dengue vaccine. The 16 antibody libraries are used for selection against live cells, live virus, and viral lysates as well as recombinant dengue antigens including envelope and NS1 proteins from the four dengue serotypes.

The identified Fab antibodies are purified for use in characterization of specificity, affinity, and competition with other Fabs and antibodies.

Key dengue antibody fragments characterized as neutralizing are converted to whole human IgG1 by subcloning coding regions into in-house mammalian expression vectors. Transfection of plasmids containing whole IgG coding sequences into mammalian cells allows production of large quantities of IgG for use in characterization and passive immunotherapy.

### EXAMPLE 5

### West Nile Virus

By applying the previously described library creation and panning technologies, antibodies that bind many of the different West Nile virus strains are isolated and produced in large quantities. As with the neutralizing antibodies described above for anthrax and VEEV, these fully human antibodies against West Nile virus are suitable for immunoprophylaxis or as immunotherapeutics.

### EXAMPLE 6

### Small Pox/Vaccinia Virus

By screening the previously described immunized human libraries against an individual antigen known to be involved in the neutralization of vaccinia, a similar panel of antibodies that bind to the antigen is obtained, and antibodies capable of neutralizing viral entry and spread in vitro and in vivo are identified. Furthermore, many variants of similar heavy chain/light chain pairs are identified by the techniques described herein, providing a range of affinities from which to select the candidates with the most desirable characteristics for testing and development. Use of more complicated mixtures of antigens for selection, such as infected cells, lysates, or virions, is also contemplated as an alternative approach. High affinity candidates derived in accordance with this can be used alone for immunoprophylaxis, without the need for affinity maturation that some other approaches may require. Alternatively, a cocktail of antibodies against specific antigens can be used, if desired. For example, Hooper et al. (2000) found that DNA vaccination utilizing genes L1R and A33R of vaccinia was more efficacious than either alone, indicating that for these two antigens, antibodies raised against both gave better protection than antibodies against one. Nowakowski et al., (Nowakowski et al., 2002) found that a mixture of three antibodies to non-overlapping epitopes derived by phage display produced potent neutralization of the botulinum neurotoxin, where each antibody alone showed little effect.

The following references are incorporated herein in their entirety by this reference:

Alibek K., Biohazard: the True Story of the Largest Covert Biological Weapons Programme in the World—Told From the Inside By the Man Who Ran It. New York, N.Y.: Random House Inc. (1999)

Barbas et al., Proc Natl Acad Sci USA 89:10164-8(1992).

Bebbington et al., Biotechnology 10:69-75(1992).

Bebbington, et al., Biotechnology 10, 169-175 (1992).

Bebbington, et al., Biotechnology 10,169-175 (1992).

Breman et al., N Engl J Med 346:1300-8(2002).

Brinton, Annu Rev Microbiol 56:371-402 (2002)

Bronze, et al., Am. J. of the Medical Sciences, 323, pp316-325 (2002).

Burioni et al., Proc Natl Acad Sci USA 91:355-9(1994).

Burton et al., Proc Natl Acad Sci USA 88:10134-7(1991).

Burton et al., Science 266:1024-7(1994).

Campbell et al., Lancet Infect Dis 2:519-29 (2002)

Casadevall, Emerg Infect Dis 8:833-41(2002).

Chen et al., Proc Natl Acad Sci USA 90:6508-12(1993).

Crowe et al., Proc Natl Acad Sci USA 91:1386-90(1994).

Davis et al., J Virol 75:4040-4047 (2001)

D'Mello et al., Meth 247:191-203(2001).

D'Mello, et al., J. Immunological Meth. 247,191-203 (2001).

Ditzel et al., J Immunol 154:893-906(1995).

Falconar, Arch. Virol. 142, 897-916 (1997).

Falkenberg, et al., J. Immunol. Methods 179, 13-29 (1995).

Fenner et al., Smallpox and its Eradication. Geneva, Switzerland: World Health Organization 1998:1460.

Franke et al., J Virol 64:5988-96 (1990).

Galmiche et al., Virology 254:71-80(1999).

Hamdan et al., Transpl Infect Dis 4:160-162 (2002)

Hooper et al., Virology 266:329-39(2000).

Hunt, et al., Virology, 185, pp281-290 (1991).

Hunt, et al., Vaccine, 13, pp281-288 (1995).

Ichihashi et al., Virology 220:491-494(1996).

Kimura-Kuroda et al., J Gen Virol 67:2663-2672 (1986)

Kinney, et al., Virology, 170, pp19-30 (1989).

Klimpel, et al., Proc Natl Acad Sci USA 89:10277-81(1992).

Klimpel, et al., Proc. Natl. Acad. Sci. USA 89,10277-10281 (1992).

Law et al., Virology 280:132-142(2001).

Little et al., Infect Immun 58:1606-13(1990).

Little, et al., Infect. Immun. 58, 1606-1613 (1990).

Maa et al., J Biol Chem 265:1662-7(1990).

Maruyama et al., J Virol 73:6024-30(1999).

Mathews, et al., J. Immunology, 129, pp2763-2767 (1982). WO02/059340A1

Miller et al., Biochemistry 38:10432-41(1999).

Miller, et al., Biochemistry 38,10432-10441 (1999).

Monath, Acad Sci 951:1-12 (2001)

Niles et al., J Virol 62:3772-8(1988).

Nir et al., Br J Exp Pathol 46:443-9 (1965)

Nowakowski et al., Proc Natl Acad Sci USA 99:11346-50(2002).

Ober et al., J Virol 76:7713-23(2002).

Paredes, et al., J. Virology, 75, pp9532-9537 (2001).

Parren et al., AIDS 9:F1-6(1995).

Parren et al., J Virol 76:6408-12(2002).

Pavlinkova et al., Cancer Immunol Immunother 49:267-75(2000).

Phillpotts, et al., Vaccine, 17, pp2429-2435 (1999).

Phillpotts, et al., Vaccine, 20, pp1497-1504 (2002).

Phinney,et al., J. Virology, 74, pp5667-5678 (2000).

Pimm et al., Nucl Med Commun 10:585-93(1989).

Pittman, et al., Vaccine, 14, pp337-343 (1996).

Ramirez et al., J Gen Virol 83:1059-67(2002).

Rodriguez et al., J Virol 56:482-8(1985).

Rodriguez et al., J Virol 69:4640-8(1995).

Roehrig, et al., Virology, 118, pp269-278 (1982).

Roehrig, et al., Virology, 142, pp347-356 (1985).

Roehrig, et al., Virology, 165, pp66-73 (1988).

Roehrig, et al., J. Clin. Microbiology, 35, pp1887-1890 (1997).

Sanna et al., Proc Natl Acad Sci USA 92:6439-43(1995).

Sanna et al., Virology 215:101-6(1996).

Schmaljohn et al., Virology 258:189-200(1999).

Schutt, et al., J. Immunol. Methods 204, 99-102 (1997).

Smallwood et al., Med J Aust 176:251-3(2002).

Van et al., Virology 271:26-36(2000).

Wang et al., 167:5273-5277 (2001)

Wang et al., Ann N Y Acad Sci 2001 b 951:325-7 (2001)

Wherle et al., Bull World Health Organ 43:669-79(1970).

Williamson et al., Proc Natl Acad Sci USA 90:4141-5(1993).

Zeitlin et al., Virology 225:213-5(1996).

It will be understood that various modifications may be made to the embodiments disclosed herein. For example, as those skilled in the art will appreciate, the specific sequences described herein can be altered slightly without necessarily adversely affecting the functionality of the antibody or antibody fragment. For instance, substitutions of single or multiple amino acids in the antibody sequence can frequently be made without destroying the functionality of the antibody or fragment. Thus, it should be understood that antibodies having a degree of homology greater than 70% to the specific antibodies described herein are within the scope of this disclosure. In particularly useful embodiments, antibodies having a homology greater than about 80% to the specific antibodies described herein are contemplated. In other useful embodiments, antibodies having a homology greater than about 90% to the specific antibodies described herein are contemplated. Therefore, the above description should not be construed as limiting, but merely as exemplifications of preferred embodiments. Those skilled in the art will envision other modifications within the scope and spirit of this disclosure.

We claim:

1. A human heterodimeric antibody or antibody fragment having a binding affinity of at least 1×10⁻⁸ M to the protective antigen of Bacillus anthracis and the ability to block binding of the protective antigen to one or more members of the group consisting of cell receptors, edema factor and lethal factor.

2. A human heterodimeric antibody or antibody fragment having a binding affinity of at least 1×10⁻⁸M to a molecule involved in anthrax infection and the ability to block binding of said molecule involved in anthrax infection to one or more members of the group consisting of cell receptors, PA63, PA63 heptamer, PA83, edema factor and lethal factor.

3. A human heterodimeric antibody or antibody fragment as in claim 1 that prevents PA63 from forming a heptamer.

4. A human heterodimeric antibody or antibody fragment as in claim 1 that prevents PA63 from binding to EF or LF.

5. A human heterodimeric antibody or antibody fragment as in claim 1 that prevents EF and/or LF from binding to the PA63 heptamer.

6. A human heterodimeric antibody or antibody fragment comprising a heavy chain variable region having a sequence selected from the group consisting of SEQ ID NO. 1 to 18.

7. A human heterodimeric antibody or antibody fragment comprising a light chain kappa region having a sequence selected from the group consisting of SEQ ID NO. 19 to 26.

8. A human heterodimeric antibody or antibody fragment comprising a light chain lambda region having a sequence selected from the group consisting of SEQ ID NO. 27 to 38.

9. A human heterodimeric antibody or antibody fragment comprising a light chain kappa region having a sequence selected from the group consisting of SEQ ID NO. 39 to 61.

10. A human heterodimeric antibody or antibody fragment comprising a light chain lambda region having a sequence selected from the group consisting of SEQ ID NO. 62 to 77.

11. A human heterodimeric antibody or antibody fragment comprising a heavy chain variable region having a sequence selected from the group consisting of SEQ ID NO. 78 to 112.

12. A method of screening antibodies comprising:
preparing a combinatorial library using RNA isolated from cells obtained from a human subject producing antibodies against one or more molecules involved in anthrax infection; and
screening the combinatorial library for an antibody having a binding affinity of at least 1×10⁻⁸ M to a molecule involved in anthrax infection and the ability to block binding of said molecule involved in anthrax infection to one or more members of the group consisting of cell receptors, PA63, PA63 heptamer, PA83 edema factor and lethal factor.

13. A method comprising:
preparing a combinatorial library using RNA isolated from cells obtained from a human subject that has been vaccinated against or exposed to a plurality of infective agents; and
screening the combinatorial library for a plurality of antibodies each having a binding affinity to a molecule involved in infection by at least one infective agent.

14. A method as in claim 13 wherein one of the plurality of antibodies identified in the step of screening is an antibody having a binding affinity of at least 1×10⁻⁸ M to a molecule involved in anthrax infection and the ability to block binding of said molecule involved in anthrax infection to one or more members of the group consisting of cell receptors, PA63, PA63 heptamer, PA83, edema factor and lethal factor.

15. A method as in claim 13 wherein one of the plurality of antibodies identified in the step of screening is an anti-Venezuelan equine encephalomyelitis virus antibody.

16. A method as in claim 13 wherein one of the plurality of antibodies identified in the step of screening is an anti-botulinum antibody.

17. A method as in claim 13 wherein one of the plurality of antibodies identified in the step of screening is an anti-West Nile virus antibody.

18. A method as in claim 13 wherein one of the plurality of antibodies identified in the step of screening is an anti-orthopox antibody.

19. A method as in claim 13 wherein one of the plurality of antibodies identified in the step of screening is an anti-dengue antibody.

20. A method as in claim 13 wherein the step of preparing a combinatorial library comprises using RNA isolated from cells obtained from a human subject that has been vaccinated against or exposed to one or more infective agents selected from the group consisting of anthrax, botulinum, smallpox, dengue, Venezuelan equine encephalomyelitis virus and West Nile virus.

21. A method as in claim 13 wherein the step of preparing a combinatorial library comprises using RNA isolated from cells obtained from a human subject that has been vaccinated against or exposed to two or more infective agents selected from the group consisting of anthrax, botulinum, smallpox, dengue, Venezuelan equine encephalomyelitis virus and West Nile virus.

22. A method as in claim 13 wherein one of the plurality of antibodies identified in the step of screening is selected from the group consisting of anti-variola antibodies, anti-monkeypox virus antibodies and anti-vaccinia virus antibodies.

23. A human heterodimeric antibody or antibody fragment having a binding affinity to a molecule involved in Venezuelan equine encephalomyelitis virus infection and the ability to neutralize Venezuelan equine encephalomyelitis virus.

24. A human heterodimeric antibody or antibody fragment comprising a heavy chain comprising a sequence selected from the group consisting of SEQ ID NO. 116 to 118.

25. A human heterodimeric antibody or antibody fragment comprising a light chain comprising a sequence selected from the group consisting of SEQ ID NO. 113 to 115.
